(19) United States
(12) Patent Application Publication
(10) Pub. No.: US 2026/0217749 A1
(43) Pub. Date: Jul. 30, 2026

(54) METAL COMPLEX COMPOUNDS AS PHOTOSENSITIZERS FOR PHOTODYNAMIC THERAPY

(71) Applicants: UNIVERSITAT DE BARCELONA, Barcelona (ES); ECOLE NATIONALE SUPERIEURE DE CHIMIE DE PARIS, Paris (FR); CENTRE NATIONAL DE LA RECHERCHE SCIENTIFIQUE, Paris (FR); PARIS SCIENCE ET LETTRES, Paris (FR)

(72) Inventors: Vicente MARCHÁN SANCHO, Barcelona (ES); Albert GANDIOSO UBIETO, Paris (FR); Gilles Albert GASSER, Paris (FR); Sergi CHUMILLAS VICEDO, Barcelona (ES); Davor CASTAÑO DURO, Barcelona (ES); Marta LÓPEZ CORRALES, Barcelona (ES); Diego ABAD MONTERO, Barcelona (ES); Eduardo IZQUIERDO GARCÍA, Barcelona (ES)

(73) Assignees: UNIVERSITAT DE BARCELONA, Barcelona (ES); ECOLE NATIONALE SUPERIEURE DE CHIMIE DE PARIS, Paris (FR); CENTRE NATIONAL DE LA RECHERCHE SCIENTIFIQUE, Paris (FR); PARIS SCIENCE ET LETTRES, Paris (FR)

(21) Appl. No.: 19/157,895

(22) PCT Filed: Feb. 20, 2024
(86) PCT No.: PCT/EP2024/054313

§ 371 Date: Aug. 19, 2025

(30) Foreign Application Priority Data
Feb. 20, 2023 (EP) 23382155.2

(51) Int. Cl. C07F 15/00; A61K 41/00

(52) U.S. Cl. CPC C07F 15/0046; A61K 41/0057

(57) ABSTRACT

Electrically neutral complex compounds of formula (I), including any stereoisomer or E/Z isomer thereof, where: M is Fe²⁺, Ru²⁺, Os²⁺, Co³⁺, Rh²⁺, Rh³⁺, Ir³⁺, Ni²⁺, Pd²⁺, Pt²⁺ or Pt⁴⁺, particularly Ru²⁺; T and T′ are H or (C1-C3)-alkyl; Lig is a bidentate ligand, particularly 2,2′-bipyridine (bpy) or 4,7-diphenyl-1,10-phenanthroline (BPhen); and A is an anion from a pharmaceutically acceptable acid, are useful in human therapy, particularly as photo sensitizers (PS) in photodynamic therapy (PDT) of cancer, skin conditions, fungal infections, or microbial infections. They are virtually non-toxic in dark conditions at therapeutic dosage, and show good to excellent photoactivity under hypoxia conditions. They are useful for PDT treatment of deep-seated hypoxic tumors by using light within the phototherapeutic window far-red to near infrared.

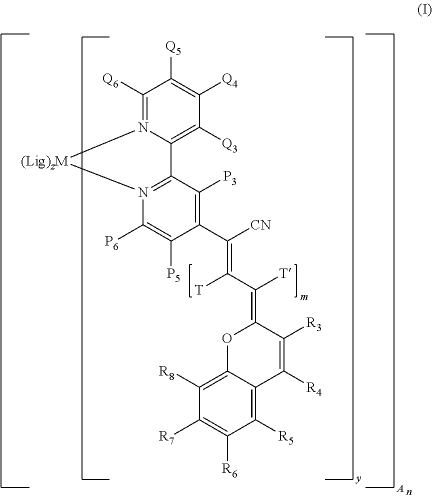

### TECHNICAL FIELD

The present invention relates to new metal complex compounds useful as photosensitizers (PS) for photodynamic therapy (PDT), and to new intermediate ligand compounds useful in the preparation of the complex compounds.

### BACKGROUND ART

Photodynamic therapy has existed for over 100 years, although in different forms. Recently, PDT has emerged as a medical intervention to treat certain types of cancer that were unable to be treated by traditional means. PDT has also been used to remedy some skin conditions (e.g. acne or port wine stains), some fungal and microbial infections, and some age-related macular degeneration (cf. S. A. McFarland et al.; “Metal-based photosensitizers for photodynamic therapy: the future of multimodal oncology?”; Current Opinion in Chemical Biology, 2020, vol. 56, pp. 23-27; and references therein).

PDT is a well-established, non-invasive modality for the destruction of tumors and/or tumor vasculature, based on the combination of three components, namely: a photosensitizer (PS), light of suitable wavelength (λ), and oxygen. PDT is delivered in a two-step procedure involving of local or systemic administration of a PS at a nontoxic dose, followed by light activation. Compared to other conventional treatment protocols, PDT exhibits some advantages for the treatment of cancer due to its noninvasive properties and to the spatiotemporal selectivity provided by light. Indeed, an important attribute of PDT is that toxicity is confined to regions where the three components (PS, light and oxygen) overlap spatiotemporally.

The PDT effect stems from excitation of the PS to produce a singlet excited state that undergoes intersystem crossing to form the reactive triplet state. The triplet state then sensitizes cytotoxic singlet oxygen (¹O₂) through type II energy transfer (predominant pathway) or participates in type I electron transfer reactions to generate other reactive oxygen species such as superoxide or hydroxyl radicals.

Photofrin® and protoporphyrin IX (PpIX), both with tetrapyrrolic ring systems, have been approved worldwide for certain cancers (cf. S. A. McFarland et al., op. cit., p. 24). While these two compounds set the standard for PDT, there is ongoing interest in developing new PSs that address some of the limitations of current PSs on clinical use.

Aqueous-soluble PSs that are prepared in high purity from straightforward syntheses are highly desirable. Certain metal complex compounds have much to offer in this respect. It is known (cf. S. A. McFarland et al., op. cit. p. 25), that some PSs containing metal ions have advanced to clinical studies, the most promising being padeliporfin (also known as TOOKAD® Soluble and WST11) and TLD-1433. Padeliporfin is a tetrapyrrolic Pd(II) complex compound used as PS in PDT to treat low-risk prostate cancer with vascular-targeted PDT. TLD-1433 is the first Ru(II) complex compound used as PS in PDT clinical studies to treat nonmuscle invasive bladder cancer. TLD-1433 is a thiophene-containing complex compound disclosed in a family of patent documents by S. A. McFarland with a priority date of 2012. This patent family includes some granted patents, such as EP 2 854 948 B1 and U.S. Pat. No. 9,676,806 B2, both of them claiming PSs which are thiophene-containing complex compounds of several metals (Mn, Mo, Re, Fe, Ru, Os, Co, Rh, Ir, Ni, Pt, and Cu).

PDT is less efficient in the treatment of deep-seated hypoxic tumors (i.e. tumor microenvironments deprived of adequate oxygen supply at the tissue level), because it is an oxygen-dependent process. On the other hand, while excitation with light of long wavelengths is usually preferred to facilitate tissue penetration, it is usually less effective in promoting type II reactive oxygen species generation (i.e. singlet oxygen) in some of the known PSs. There is ongoing interest in developing new PSs based on metal complex compounds for PDT which address some of the limitations of known PSs, particularly with operability within the far-red and near infrared region (NIR) of the electromagnetic spectrum and under hypoxia conditions.

### SUMMARY OF INVENTION

An aspect of the present disclosure relates to the provision of compounds of formula I or IV, including stereoisomers and E/Z isomers,
wherein:
M is a metal cation selected from the group consisting of: Fe²⁺, Ru²⁺, Os²⁺, Co³⁺, Rh²⁺, Rh³⁺, Ir³⁺, Ni²⁺, Pd²⁺, Pt²⁺, and Pt⁴⁺;
y=1, 2, or 3; and z=0, 1, or 2; on the proviso that: y+z=2 when M is Ni²⁺, Pd²⁺ or Pt²⁺, and y+z=3 when M is Fe²⁺, Ru²⁺, Os²⁺, Co³⁺, Rh²⁺, Rh³⁺, Ir³⁺, or Pt⁴⁺;
m=0, 1, 2, 3, or 4;
radicals T and T′ at each occurrence are each one a radical independently selected from the group consisting of: H and (C1-C3)-alkyl;

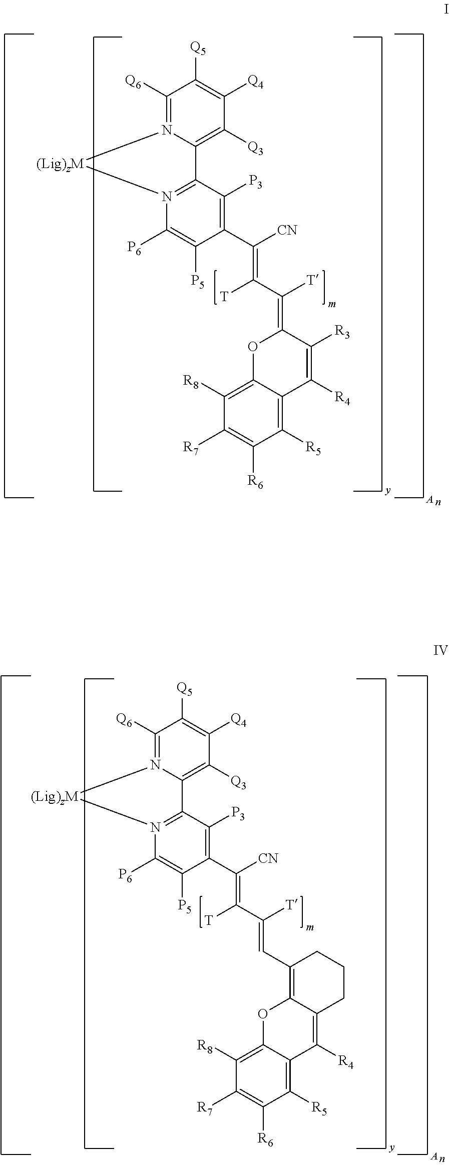

radicals P₃, P₅, P₆, Q₃, Q₄, Q₅, Q₆, R₃ (the definition of R₃ is only applicable to formula I as formula IV does not comprise this specific radical), R₅, R₆, and R₈ at each occurrence are each one a radical independently selected from the group consisting of: H, (C1-C3)-alkyl, (C3-C6)-cycloalkyl, CH═CHR, CF₃, CHF₂, CH₂F, CF₂CF₃, F, Cl, Br, I, OR, C(═O)OR, O(C═O)R, C(═O)NR, NRC(═O)R′, NRR′, phenyl, mono-substituted phenyl, di-substituted phenyl, and tri-substituted phenyl; wherein substituents on the phenyl ring are attached to any of the possible substitution positions, and they are independently selected from the group consisting of: F, Cl, Br, I, NO₂, (C1-C3)-alkyl, OH, O[(C1-C3)-alkyl], NH₂, NH[(C1-C3)-alkyl], and N[(C1-C3)-alkyl]₂; radical R₇ is H, NO₂, OR, NRR′, N(CH₂COOH)₂, N(CH₂CH₂SO₃H)₂, N(CH₂CONHCH₂CH₂NMe₂)₂, or: an unsubstituted, a mono-(R)-substituted, a di-(R,R′)-substituted, or a tri-(R,R′,R″)-substituted radical of each one of the heterocyclic radicals 1-aziridinyl, 1-azetidinyl, 1-pyrrolidinyl, 1-piperidinyl, 4-morpholinyl, or 1-piperazinyl; alternatively R₆, R₇ and R₈ form together the bicycle system of the accompanying formula, wherein V₁, V₂, V₃, V₄, V₅, V₆, V₇, V₈, V₉, V₁₀, V₁₁, and V₁₂ are each one a radical independently selected from the group consisting of H and (C1-C3)-alkyl;

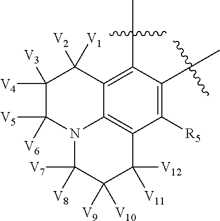

radical R₄ is H, (C1-C3)-alkyl, (C3-C6)-cycloalkyl, CF₃, CHF₂, CH₂F, CF₂CF₃, F, Cl, Br, I, OR, C(═O)OR, O(C═O)R, C(═O)NR, NRC(═O)R′, NRR′, phenyl, a mono-(R)-substituted phenyl, a di-(R, R′)-substituted phenyl, a tri-(R, R′, R″)-substituted phenyl, or a radical CE₁E₂E₃; wherein: E₁ and E₂ are radicals independently selected from the group consisting of: H, F, Cl, Br, I, OR, NRR′, NO₂, (C1-C3)-alkyl, phenyl, and a mono-(R)-substituted phenyl; and E₃ is OH, O[(C1-C3)-alkyl], or a radical of the accompanying formula wherein p=0, 1, 2, 3 or 4; and D₁ and D₂ at each occurrence are radicals independently selected from the group consisting of H, O[CH₂]_{q}—X, S[CH₂]_{q}—X, and N[[CH₂]_{q}—X]₂, being q=1 or 2, and X=F, Cl, Br or I.

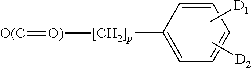

or a radical of the accompanying formula wherein p=1, 2, 3 or 4; and D3 is a radical independently selected from the group consisting of H, H(C═O), [(C1-C3)-alkyl](C═O), (OH)₂P(═O)O—CH₂—O(C═O), (PhO)(OH)P(═O)

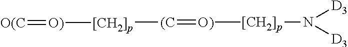

Lig at each occurrence is a bidentate ligand independently selected from the group consisting of ligands of accompanying formulae: Lig 1, Lig 2, Lig 3, Lig 4, Lig 5, Lig 6, Lig 7, Lig 8, Lig 9, Lig 10, Lig 11, Lig 12, and Lig 13; wherein G is either an N atom, or a C atom with one negative charge that is formed in situ from a C—H when a H⁺ is lost in the formation of the carbon-metal bond; and wherein R₁₀, R₁₁, R₁₂, R₁₃, R₁₄, R₁₅, R₁₆, R₁₇, R₁₈, R₁₉, R₂₀ and R₂₁ at each occurrence is each one a radical independently selected from the group consisting of: H, (C1-C3)-alkyl, phenyl, and mono-, di- and tri-substituted phenyl, substituents attached to the phenyl ring being independently selected from F, Cl, Br, I, OR and NRR′; and
A is an anion from a pharmaceutically acceptable acid; n being an integer or fractional number whereby the compound of formula I is electrically neutral;
wherein R, R′ and R″ at each one of the above-mentioned occurrences are each one a radical independently selected from the group consisting of: H, F, Cl, Br, I, NO₂, (C1-C3)-alkyl, OH, Of(C1-C3)-alkyl], NH₂, NH[(C1-C3)-alkyl], and N[(C1-C3)-alkyl]₂.

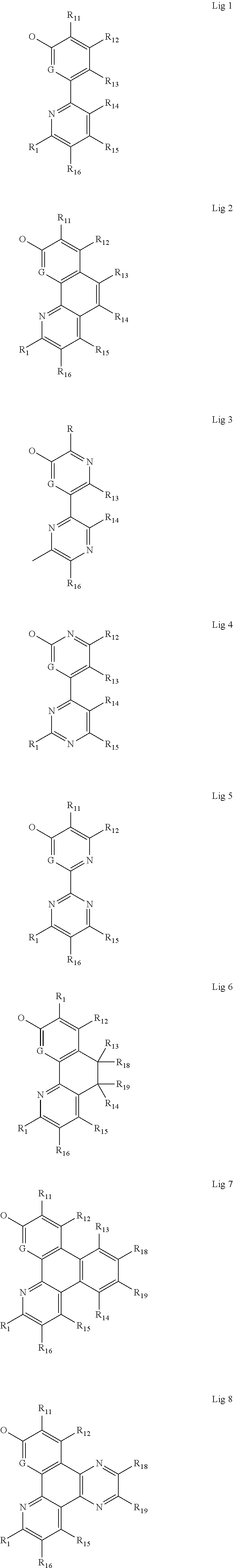

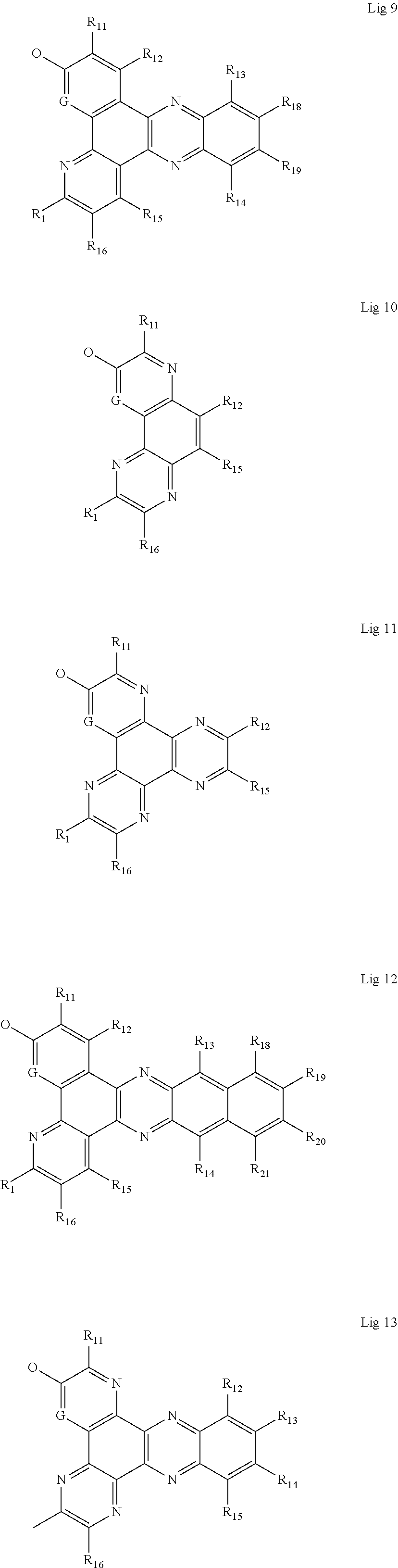

As used herein, the term “stereoisomer” is a general term for all isomers of individual molecules that differ only in the orientation of their atoms in space, and include enantiomers and diastereomers. When the compounds have chiral centers, they can exist in different stereoisomeric forms, such as enantiomeric or diastereomeric forms. Thus, any given compound referred to herein is intended to represent any one of a racemate, one or more enantiomeric forms, and one or more diastereomeric forms. All the stereoisomers, including enantiomers and diastereoisomers of the compounds referred to herein, and mixtures thereof (including racemic mixtures, enantiomerically enriched mixtures, and diastereomerically enriched mixtures), are considered within the scope of the present disclosure. The disclosure further contemplates any E/Z possible isomers and their mixtures.

It is noted that, in a preferred embodiment of the present invention, formula IV can be obtained from formula I, when in formula I m≥1 (such 1, 2, 3 or more), radical R₃ being CH₂ forms a 6-membered ring with the T′ radical closest to the coumarin backbone provided that T′=CH₂. It is further noted that, in another preferred embodiment of the present invention, formula V can be obtained from formula II, when in formula II m≥1 (such 1, 2, 3 or more), radical R₃ being CH₂ forms a 6-membered ring with the T′ radical closest to the coumarin backbone provided that T′=CH₂.

Generally, complex compounds of formula I or IV can be obtained by reacting—in a solvent or a solvent mixture—the corresponding intermediate compounds of formula II or V (respectively), including the corresponding stereoisomers or E/Z isomers thereof, with the corresponding reactant compounds of formula III, wherein m, n, y, z, P₃, P₅, P₆, Q₃, Q₄, Q₅, Q₆, R₃, R₄, R₅, R₆, R₇, R₈, T, T′, M, Lig and A, are as defined above. As intermediate compounds of formula II or V are novel, another aspect of the present disclosure relates to the provision of these compounds, which are useful in the preparation of complex compounds of formula I or IV.

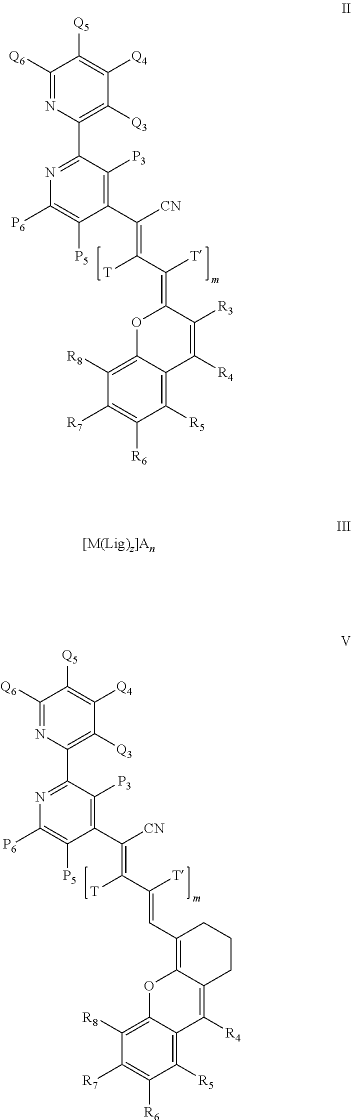

In general, reactant compounds of formula III and intermediate compounds of formula II or V are reacted in molar amount ratios which substantially correspond to the stoichiometry determined by the values of z and y, although an excess of III may be recommended in some cases to increase yield. In some cases, an intermediate compound is obtained with a different anion A′, and a final anion-exchange reaction (e.g. with an ion-exchange resin) is carried out.

As illustrated by the results shown in the description of embodiments section, metal complex compounds of formula I or IV are useful as PSs in anticancer PDT. For example, complex compounds of formulas Ia and Ic show very good phototoxicity results towards CT-26 mouse colon carcinoma cancer cell line.

Thus, another aspect of the present disclosure relates to the provision of pharmaceutical compositions comprising a therapeutically effective amount of a complex compound of formula I or IV, together with appropriate amounts of excipients, carriers or vehicles.

Complex compounds of formula I are virtually non-toxic in dark conditions at therapeutic dosage. For example, both complex compound Ia and complex compound Ic are non toxic in dark conditions with IC₅₀ values over 250 μM, but become highly cytotoxic after irradiation with monochromatic visible light. As shown in Table 1, IC₅₀ values for both compounds were found in the low nanomolar range (8.2 nM for compound Ia at 540 nm, and 7.4 nM for compound Ic at 645 nm) with very good PI values (>30487 and >33783, respectively). For comparison purpose, results reported for TLD-1433, a PS that is in clinical development, are as follows: the IC₅₀ value of TLD-1433 in SK-MEL-28 melanoma cells is in the low micromolar range (2.3 μM) after red light irradiation (625 nm), and it shows a moderate PI value (cf. S. Monro et al.; “Transition metal complexes and photodynamic therapy from a tumor-centered approach: challenges, opportunities, and highlights from the development of TLD-1433”; Chem. Rev. 2019, vol. 119, pp. 797-828).

Compound of formula Ic shows a good phototoxicity profile with highly penetrating NIR light (740 nm), both in the CT-26 cancer cell line (IC₅₀=0.76 μM, PI>329) and in its human analogue HT-29 cancer cell line (IC₅₀=0.35 μM, PI>714); these results are better than those corresponding to Protoporphyrin IX, which is the drug used as control. As illustrated by results in Table 6, similar results are obtained in other human cancer cell lines (A-549 and A-2780). Similarly, as shown in Table 5 and Table 8, compounds of formula Im, In, Io and IVa were found phototoxic upon irradiation with NIR light (740 and 770 nm) in CT-26 cancer cell line (e.g. IC₅₀=0.17 μM for Im at 740 nm, PI>588).

Complex compounds of formula I or IV show good to excellent photoactivity under hypoxia conditions. For example, compounds Ia and Ic show excellent photoactivity under hypoxia conditions, compound Ia being particularly toxic when irradiated at 540 nm, having an excellent PI over 7143. Compound Ic show PI values around 2900-3300 in the light-window of 540-670 nm. Compound Im show submicromolar IC₅₀ values (PI>168) when irradiated with NIR light (740 nm) under hypoxia. Such results illustrate that compounds or IV are useful for the PDT treatment of deep-seated hypoxic tumors by using light within the phototherapeutic window far-red to NIR.

Aspects of the present disclosure relate to complex compounds of formula I or IV as for use in human therapy, particularly for use as photosensitizer in photodynamic therapy of a human condition; more specifically the human condition is a cancer, a skin condition, a fungal infection or a microbial infection. These aspects also relate to preparation processes of medicaments for photodynamic therapy in humans of cancer, skin conditions, fungal infections or microbial infections, comprising the use of complex compounds of formula I or IV, preferably in the form of nanoformulations. In other words, it can be said that complex compounds of formula I or IV are useful in methods of treatment by photodynamic therapy, of cancer, skin conditions, fungal infections or microbial infections in humans.

Throughout the present disclosure and claims the word “comprise” and variations of the word, are not intended to exclude other technical features, additives, components, or steps. Furthermore, the word “comprise” encompasses the case of “consisting of”. Additional objects, advantages and features will become apparent to those skilled in the art upon examination of the disclosure, or may be learned by practice of the disclosure. The following examples are provided by way of illustration, and they are not intended to be limiting.

### BRIEF DESCRIPTION OF THE FIGURES

FIG. 1. Top: Monitored growth of 3D CT-26 multicellular tumor spheroids (MCTSs) over 7 days compared to the initial sizes at increasing concentrations of Ic. Data expressed as mean±SD from three independent measurements. Control: non-treated MCTSs, irradiated for 1 h with 645 nm light (2.50 mW·cm⁻², 9.00 J·cm⁻²). Treated MCTSs were incubated with Ic for 36 h and irradiated for 1 h with 645 nm light (2.50 mW·cm⁻², 9.00 J·cm⁻²). Bottom: Micrographs of 3D CT-26 MCTSs taken over 7 days (Scale bar=1000 μm).

FIG. 2. Cellular uptake of complex compound Ic. Single confocal planes of HeLa cells incubated with the compound (10 μM) for 30 min at 37° C., imaged at t=0 and after 2 min of first observation. Excitation was performed with three different laser lines (405, 458 and 514 nm). White arrows point out mitochondria and white arrowheads vesicle staining. Black arrowheads on the right column point out cell blebbings. Scale bar: 20 μm. LUT for compound images: Fire. Left and right columns: merge of compound and brightfield images.

FIG. 3. Co-localization study of Ic with Mitoview 650. Single confocal planes of HeLa cells incubated with the compound (10 μM, green) and Mitoview (0.1 μM, red). Left: Overlay of the two staining. Center: Ic′ signal. Right: Mitoview signal. White arrows and arrowheads point out positive and negative (vesicles staining in compound images) colocalization, respectively. Scale bar: 20 μm.

FIG. 4. Mean of concentrations of Ic in albino swiss adult male CD1 mice plasma at different times after IP administration at 5 mg/Kg (left) and pharmacokinetic parameters of Ic in mouse plasma (right).

FIG. 5. Body weight (g) (left) and food consumption (g)/animal (right) during the 5-day period experimental protocol. CD1 mice were intraperitoneally treated with vehicle and complex compound Ic (10 mg/kg or 30 mg/kg) on day 1 and sacrifice on day 5. Results are expressed as mean±SD (n=3 Males; n=3 Females).

FIG. 6. Organ weight (mg)/Body weight (g) ratio of animals intraperitoneally treated with vehicle and complex compound Ic (10 mg/kg or 30 mg/kg) on day 1 and sacrifice on day 5. Results are expressed as mean±SD (n=3 Males; n=3 Females). *p<0.05 vs. male or female vehicle group (1-way ANOVA and Bonferroni post-test).

FIG. 7. In vivo PDT efficacy study of Ic in female BALB/c mice bearing subcutaneous CT-26 syngeneic colon tumors after IT administration. Irradiation was performed with 660 nm LED light (15 min, 100 mW/cm²). Tumor growth inhibition curves (left) and average tumor weights (right) of the treated tumor-bearing mice (D: dark group; L: irradiated group; xₙ: number of irradiations). Data are expressed as mean±SEM of five parallel mice (n=5). Average tumor weight data were analyzed using a one-way ANOVA followed by Bonferroni's multiple comparison test (Asterisks: *p<0.05, **p<0.001).

### DESCRIPTION OF EMBODIMENTS

In particular embodiments of complex compounds of formula I or IV, the anion A is Cl⁻, Br⁻, PF₆⁻, PF₄⁻, BF₄⁻, ClO₄⁻, CF₃SO₃⁻, SO₄²⁻, CF₃COO⁻, acetate, formate, and oxalate. In particular embodiments A is Cl⁻.

In particular embodiments of complex compounds of formula I or IV, y=1, z=2, and the metal cation M is Fe²⁺, Ru²⁺, Os²⁺, Co³⁺, Rh²⁺, Rh³⁺, Ir³⁺, or Pt⁴⁺. In particular embodiments, the metal cation M is Ru²⁺.

In particular embodiments of complex compounds of formula I or IV, the bidentate ligand Lig is Lig 1 or Lig 2; and each one of the radicals R₁₀, R₁₁, R₁₂, R₁₃, R₁₄, R₁₅, R₁₆, R₁₇, R₁₈, R₁₉, R₂₀ and R₂₁ at each occurrence is H, methyl or phenyl. In particular embodiments, G is N. In particular embodiments, the bidentate ligand Lig is 2,2′-bipyridine (bpy) or 4,7-diphenyl-1,10-phenanthroline (BPhen).

In particular embodiments, some complex compounds of formula I or IV are obtained by reaction between intermediate compounds of formula II or V and reactant compounds of formula III, wherein M=Ru²⁺, A=Cl⁻, z=2, y=1, n=2, Lig=2,2′-bipyridine (bpy) or 4,7-diphenyl-1,10-phenanthroline (BPhen), the solvent mixture is a mixture of ethanol and water, and the molar amount ratio of compound III to compound II or V is 1.0 or slightly higher.

In particular embodiments of complex compounds of formula I or IV, each one of the radicals P₃, P₅, P₆, Q₃, Q₄, Q₅, Q₆, R₃ (the definition of R₃ is only pertinent for formula as formula IV fails to comprise this radical), R₅, R₆, and R₈ at each occurrence is H or (C1-C3)-alkyl; and the radical R₇ is H, N[(C1-C3)-alkyl)]₂ or NOR; alternatively the radicals R₆, R₇ and R₈ form together the bicycle system of the formula shown in claim 1, wherein each one of V₁, V₂, V₃, V₄, V₅, V₆, V₇, V₈, V₉, V₁₀, V₁₁, and V₁₂ is H.

In particular embodiments of complex compounds of formula I or IV, the radical R₄ is H, (C1-C3)-alkyl, CF₃, CH(CH₃)OH, CH(CH₃)O(C═O)—[CH₂]₃-Ph, CH(CH₃)O(C═O)—[CH₂]₂—(C═O)—CH₂—NH₂, or a radical selected from the group consisting of:

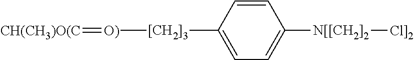

It is noted that, in a preferred embodiment of the present invention, formula IV can be obtained from formula I, when in formula I m≥1 (such 1, 2, 3 or more), radical R₃ being CH₂ forms a 6-membered ring with the T′ radical closest to the coumarin backbone provided that T′=CH₂. It is further noted that, in another preferred embodiment of the present invention, formula V can be obtained from formula II, when in formula II m≥1 (such 1, 2, 3 or more), radical R₃ being CH₂ forms a 6-membered ring with the T′ radical closest to the coumarin backbone provided that T′=CH₂.

In particular embodiments m=0, 1 or 2. In particular embodiments, the complex compounds of formula I or IV have one of the accompanying formulas Ia, Ib, Ic, Id, Ie, If, Ig, Ih, Im, In, Io, Ip, Iq, IVa or IVb:

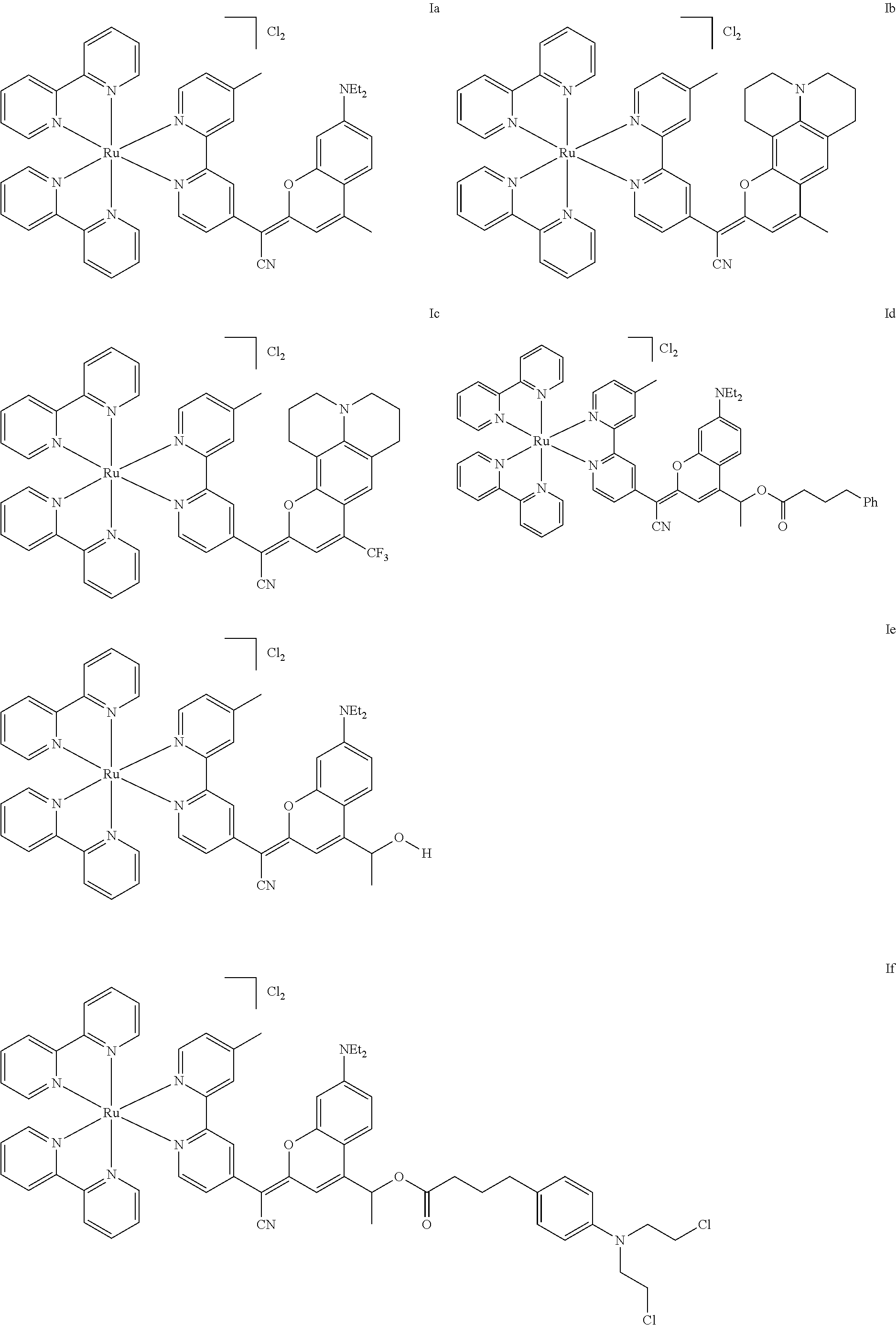

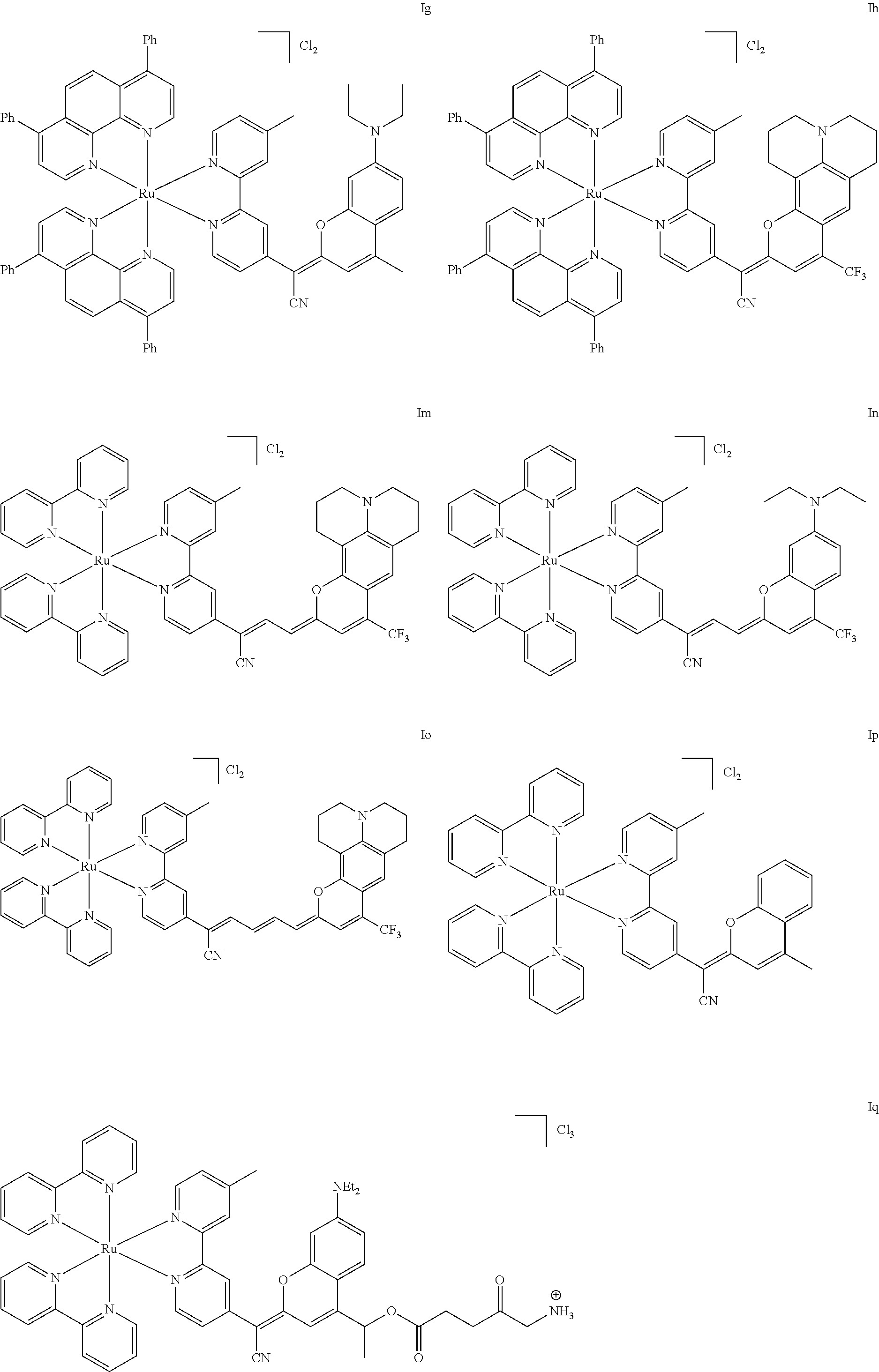

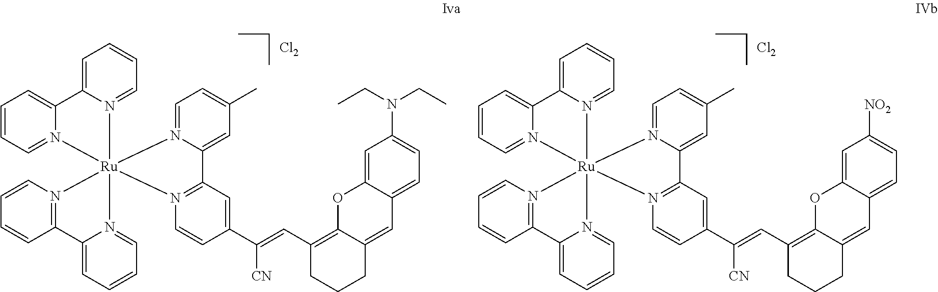

In particular embodiments the intermediate compounds of formula II (IIa, IIb, IIc, IId, IIe, IIf, IIm, IIn, IIo, IIp, IIq, IIr) or V (Va or Vb) have one of the accompanying formulas IIa, IIb, IIc, IId, Ile, IIf, IIm, IIn, IIo, IIp, IIq, IIr, Va or Vb.

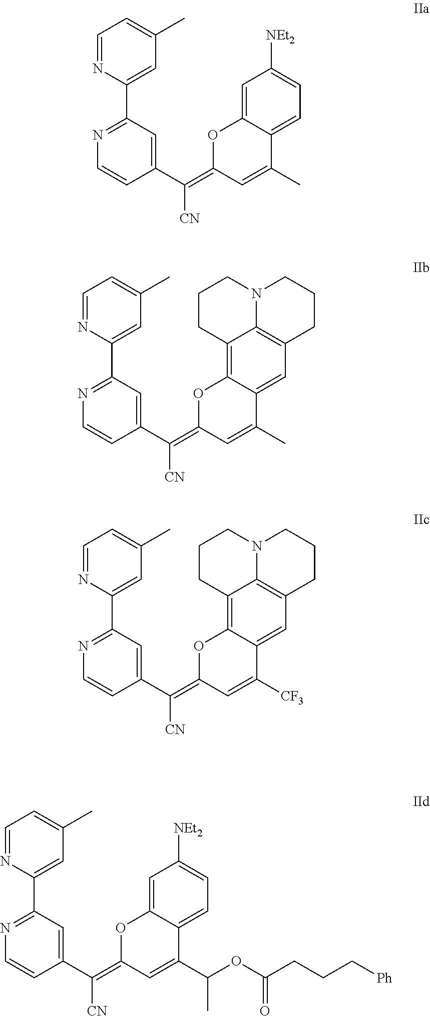

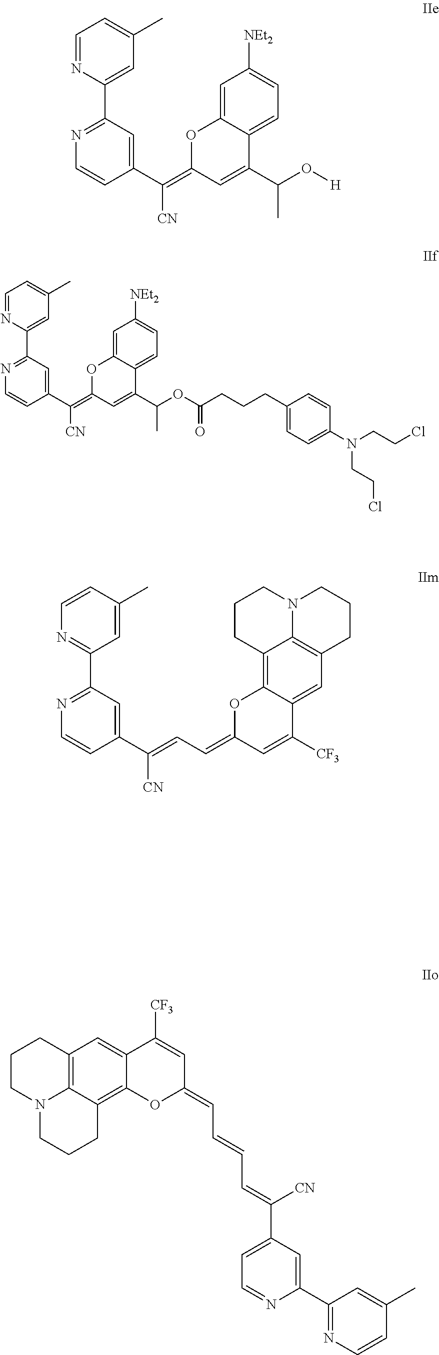

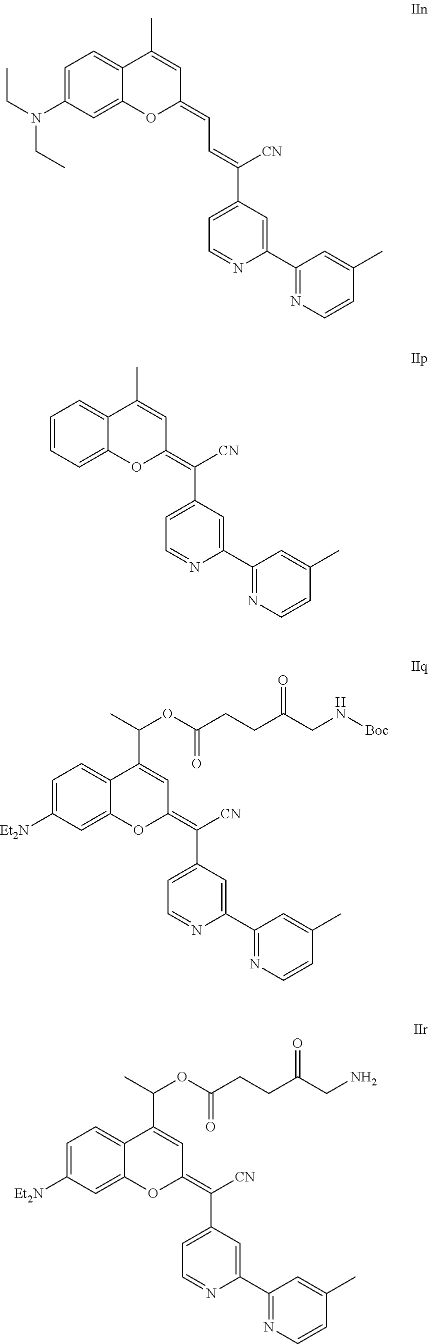

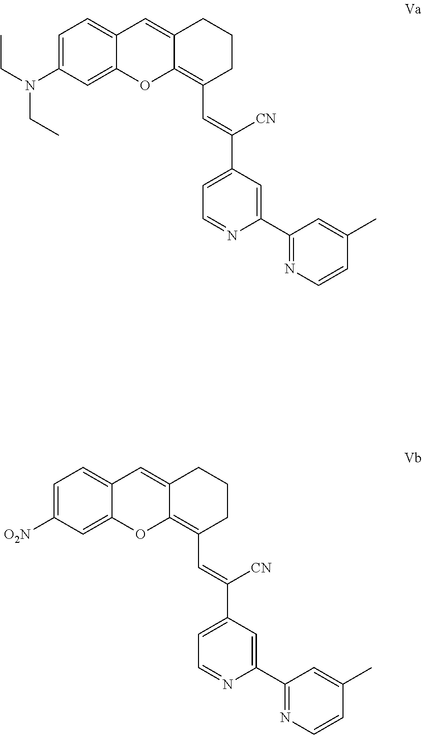 [SMILES: CCN(CC)c1ccc2c(c1)OC1=C(/C=C(\C#N)c3ccnc(-c4cc(C)ccn4)c3)CCCC1=C2 ; Cc1ccnc(-c2cc(/C(C#N)=C/C3=C4Oc5cc([N+](=O)[O-])ccc5C=C4CCC3)ccn2)c1]

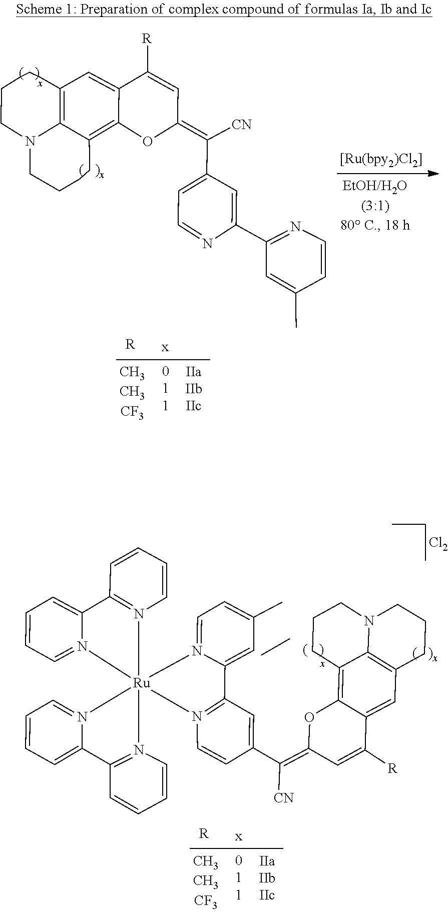

Complex compound of formula Ia. Ligand compound of formula Ila (31 mg, 0.073 mmol) and [Ru(bpy)₂Cl₂] (37 mg, 0.076 mmol) were dissolved in 3 mL of a 3:1 (v/v) solution of EtOH/H₂O. The reaction mixture was stirred overnight at 80° C. and analyzed by HPLC-MS to confirm the formation of the product. The solution mixture was evaporated to dryness and the resulting aqueous solution was lyophilized and the product was isolated by silica column chromatography starting with hexanes and increasing the eluent polarity with DCM first (0-100%) and then with MeOH (0-20%). 41 mg of a red-maroon solid were obtained (yield: 89%), identified as complex compound of formula Ia. TLC: Rf (35% MeOH in DCM) 0.4. HR-ESI MS (ESI): m/z 418.1265 calc. for [C₄₇H₄₂N₈ORu]²⁺: 418.1257.

Complex compound of formula Ib. Ligand compound of formula IIb (34 mg, 0.076 mmol) and [Ru(bpy)₂Cl₂] (38 mg, 0.078 mmol) were dissolved in 3 mL of a 3:1 (v/v) solution of EtOH/H₂O. The reaction mixture was stirred overnight at 80° C. and analyzed by HPLC-MS to confirm the formation of the product. The solvent was evaporated to dryness and the product was isolated by silica column chromatography starting with hexanes and increasing the eluent polarity with DCM first (0-100%) and then with MeOH (0-20%). 46 mg of a dark-maroon solid were obtained (yield: 65%), identified as complex compound of formula Ib. TLC: Rf (1:9 MeOH/DCM) 0.5. HR-ESI MS (ESI): m/z 430.1258, calc. for [C₄₉H₄₂N₈ORu]²⁺: 430.1257.

Complex compound of formula Ic. Ligand compound of formula IIc (31.6 mg, 0.063 mmol) and [Ru(bpy)₂Cl₂] (37.4 mg, 0.077 mmol) were dissolved in 2 mL of a 3:1 (v/v) solution of EtOH/H₂O. The reaction was stirred overnight at 80° C. and analyzed by HPLC-MS to confirm the formation of the product. The solvent was evaporated to dryness and the product was isolated by silica column chromatography starting with hexanes and increasing the eluent polarity with DCM first (0-100%) and then with MeOH (0-16%). 51.4 mg of a dark-purple solid were obtained, identified as complex compound of formula Ic (yield: 87%). TLC: Rf (30% MeOH in DCM) 0.4. HR-ESI MS (ESI): m/z 457.1102 calc. for [C₄₉H₃₉N₈OF₃Ru]²⁺: 457.1115.

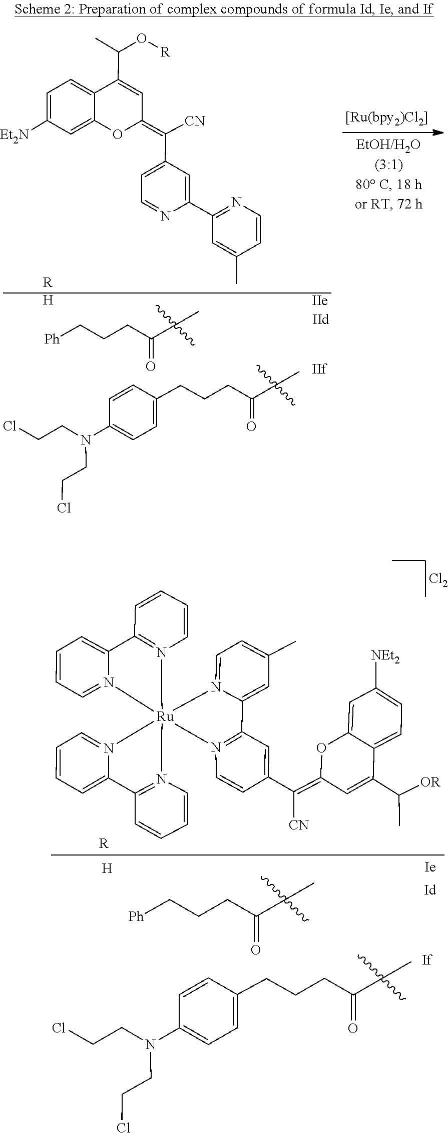

Complex compound of formula Id. Ligand compound of formula IId (57.7 mg, 0.096 mmol.) and [Ru(bpy)₂Cl₂] (55.7 mg, 0.115 mmol) were dissolved in 6 mL of a 3:1 (v/v) solution of EtOH/H₂O. A few drops of DCM (0.6 mL) were used to fully dissolve the reagents. The reaction mixture was heated to 80° C. and it was kept under reflux overnight. The solvent was evaporated to dryness and the product was purified by flash chromatography (Puriflash system; DCM in hexanes 0-100% first and then MeOH in DCM 0-44%, Silica column-PF-30SIHP-F0012) affording 34 mg of a red-maroon solid (yield: 34%), identified as complex compound of formula Id. TLC: Rf (30% MeOH in DCM) 0.43. HR-ESI MS (ESI): m/z 506.1689 calc. for [C₅₈H₅₄N₈O₃Ru]²⁺: 506.1681.

Complex compound of formula Ie. Ligand compound of formula Ile (21.35 mg, 0.047 mmol) and [Ru(bpy)₂Cl₂] (27.32 mg, 0.056 mmol) were dissolved in a 3:1 (v/v) mixture of EtOH and H₂O (2 mL). A few drops of DCM (0.3 mL) were also added to fully dissolve the reagents. The reaction mixture was left to react overnight at 80° C. After evaporation of the solvent under reduced pressure, a product was isolated by semipreparative RP-HPLC (gradient from 10-100% B in 30 min; A, 0.05% TFA in H₂O; B, 0.1% TFA in ACN; flow rate, 3 mL). After lyophilization, 16.4 mg of a red solid (yield: 32%) were obtained. The chloride salt of the compound, identified as complex compound of formula Ie, was obtained by using an Amberlite IRA402 chloride resin. LR-ESI MS (ESI): m/z 433.34 calc. for [C₄₈H₄₄N₈O₂Ru]²⁺: 433.1315.

Complex compound of formula If. [RuCl₂(bpy)₂] (18.4 mg, 0.038 mmol) and AgNO₃ (6.5 mg, 0.038 mmol) were dissolved in 1:1 (v/v) mixture of MeOH and Milli-Q water (2.4 mL) and stirred for 4 h at 90° C. protected from light. Then, a solution of ligand compound I If (28 mg, 0.038 mmol) in THE (1.2 ml) was added, and the reaction mixture allowed to react for 72 h at room temperature. Finally, KPF₆ (69.9 mg, 0.38 mmol) was added and left to react overnight. After evaporation of the solvent under reduced pressure, the product was isolated by semipreparative RP-HPLC (gradient from 50-100% B in 30 min; A, 0.05% TFA in H₂O; B, 0.1% TFA in ACN; flow rate, 3 mL). After lyophilization, 18.8 mg of a red solid (yield: 36%). TLC: R_{f} (30% MeOH in DCM)=0.48. The chloride salt of the compound, identified as complex compound of formula If, was obtained by using an Amberlite IRA402 chloride resin. LR-ESI MS (ESI): m/z 575.81 calc. for [C₆₂H₆₁Cl₂N₉O₃Ru]²⁺: 575.6659.

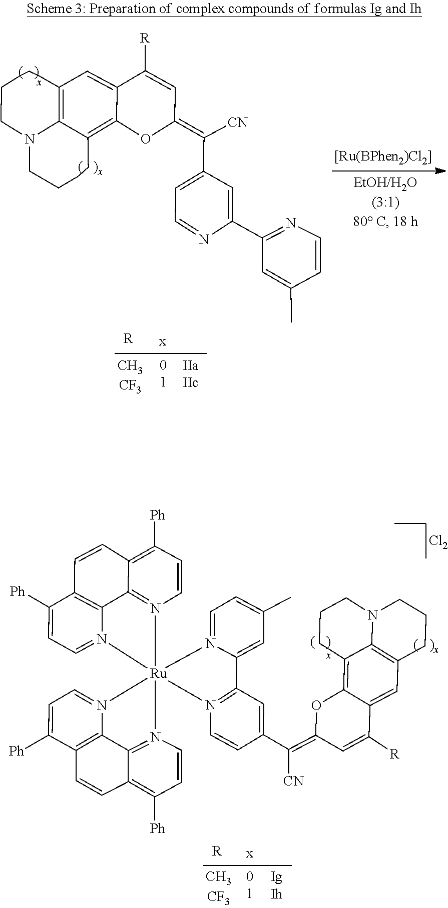

Complex compound of formula Ig. Ligand compound of formula Ila (20.4 mg, 0.048 mmol) and [Ru(BPhen)₂Cl₂]) (49.5 mg, 0.059 mmol) were dissolved in 3 mL of a 3:1 (v/v) solution of EtOH/H₂O. The reaction mixture was stirred overnight at 80° C. and analyzed by HPLC-MS to confirm the formation of the product. The solution mixture was evaporated to dryness and the product was isolated by silica column chromatography starting with 100% DCM and increasing the eluent polarity with MeOH (0-6%). 37.3 mg of a red solid were obtained (yield: 63%), identified as complex compound of formula Ig. TLC: Rf (10% MeOH in DCM) 0.4. HR-ESI MS (positive mode): m/z 594.1892 calc. for [C₇₅H₅₈N₈ORu]²⁺: 594.1888.

Complex compound of formula Ih. Ligand compound of formula IIc (11.0 mg, 0.022 mmol) and [Ru(BPhen)₂Cl₂] (41.9 mg, 0.050 mmol) were dissolved in 8 mL of a 3:1 (v/v) solution of EtOH/H₂O. The reaction mixture was stirred overnight at 80° C. and analyzed by HPLC-MS to confirm the formation of the product. The solution mixture was evaporated to dryness and the product was isolated by silica column chromatography starting with hexanes and increasing the eluent polarity with DCM first (0-100%) and then with MeOH (0-26%). 23.7 mg of a red-maroon solid were obtained (yield: 81%), identified as complex compound of formula Ih. TLC: Rf (10% MeOH in DCM) 0.3. HR-ESI MS (positive mode): m/z 633.1740 calc. for [C₇₇H₅₅F₃N₈ORu]²⁺: 633.1741.

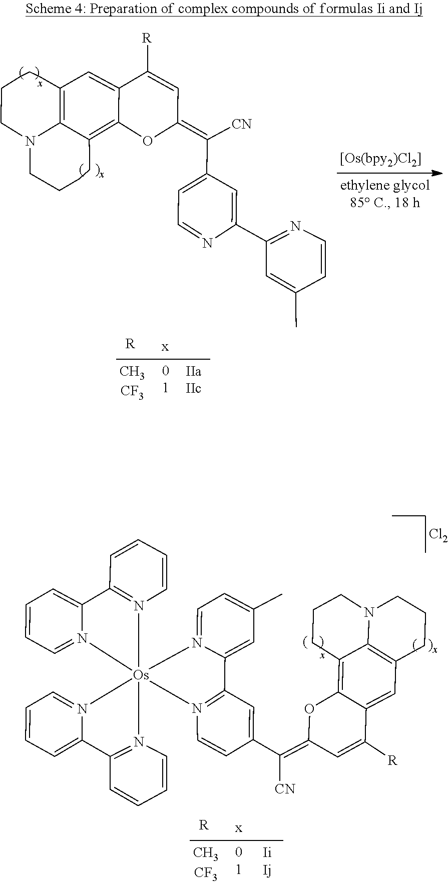

Complex compound of formula II. To a solution of [Os(bpy)₂Cl₂] (10 mg, 0.017 mmol) in ethylene glycol was added the the ligand compound of formula IIa (8 mg, 0.019 mmol). The mixture was heated at 85° C. under N₂ for 18 h. The solution was cooled down to room temperature. A saturated aqueous solution of ammonium hexafluorophosphate was added, and the resulting dark purple precipitate was then isolated by filtration, washed with water, diethyl ether, and dried under vacuum. The crude product was then purified by column chromatography (silica gel, 0-15% MeOH in CH₂Cl₂). After conversion of the PF₆ salt to chloride salt using an Amberlite IRA 410 chloride resin, 8 mg of a dark purple solid were obtained (42% yield). TLC: Rf (10% MeOH in CH₂Cl₂) 0.45. HR-ESI MS (positive mode): m/z 463.1542 calc. for [C₄₇H₄₂N₈OOs]²⁺: 463.1542.

Complex compound of formula Ij. To a solution of [Os(bpy)₂Cl₂] (14 mg, 0.024 mmol) in ethylene glycol was added the ligand compound of formula IIc (12 mg, 0.028 mmol). The mixture was heated at 85° C. under N₂ for 18 h. The solution was cooled down to room temperature. A saturated aqueous solution of ammonium hexafluorophosphate was added, and the resulting dark purple precipitate was then isolated by filtration, washed with water, diethyl ether, and dried under vacuum. The crude product was then purified by column chromatography (silica gel, 0-15% MeOH in CH₂Cl₂). After conversion of the PF₆ salt to chloride salt using an Amberlite IRA 410 chloride resin, 7 mg of a dark purple solid were obtained (28% yield). Characterization: TLC: Rf (10% MeOH in CH₂Cl₂) 0.40. HR-ESI MS (positive mode): m/z 502.1402 calc. for [C₅₁H₄₅F₃N₈OOs]²⁺: 502.1401.

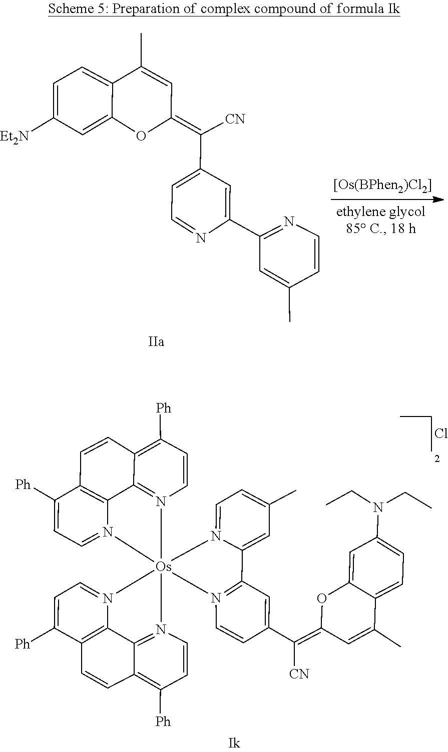

Complex compound of formula Ik. To a solution of [Os(BPhen)₂Cl₂] (30 mg, 0.032 mmol) in degassed ethylene glycol was added the ligand compound of formula Ila (14.8 mg, 0.035 mmol). The mixture was heated at 110° C. under N₂ for 18 h. The solution was cooled down to room temperature. A saturated aqueous solution of ammonium hexafluorophosphate was added, and the resulting dark purple precipitate was then isolated by filtration, washed with water, diethyl ether, and dried under vacuum. The crude product was purified first by column chromatography (silica gel, 0-15% MeOH in CH₂Cl₂), and then it was further purified by preparative HPLC (gradient from 0 to 100% B in 30 min, flow rate: 10 mL min⁻¹, Rₜ=15.4 min). After conversion of the PF₆ salt to chloride salt using an Amberlite IRA 410 chloride resin, 9 mg of a dark purple solid were obtained (22% yield). TLC: Rf (10% MeOH in CH₂Cl₂) 0.51. HR-ESI MS (positive mode): m/z 639.2181 calc. for [C₇₅H₅₈N₈OOs]²⁺: 639.2169.

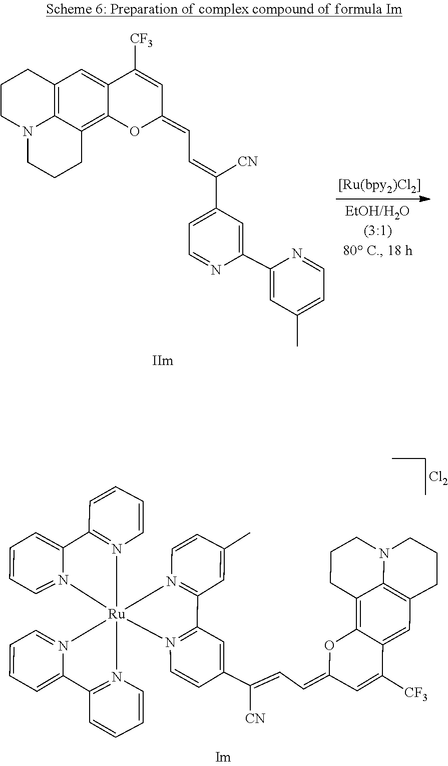

Complex compound of formula Im. Ligand compound of formula IIm (31.6 mg, 0.060 mmol) and [Ru(bpy)₂Cl₂] (37.4 mg, 0.077 mmol) were dissolved in a 3:1 (v/v) solution of EtOH/H₂O (2 mL) and the reaction mixture was stirred overnight at 90° C. The solvent was evaporated to dryness and the product was purified by silica column chromatography starting with hexanes and increasing the eluent polarity with DCM first (0-100%) and then with MeOH (0-11%). 10 mg of a blue solid were obtained (yield: 17%), identified as complex compound of formula Im. TLC: Rf (30% MeOH in DCM) 0.3. HR-ESI MS (ESI): m/z 470.1193 calc. for [C₅₁H₄₁F₃N₈ORu]²⁺: 470.1199

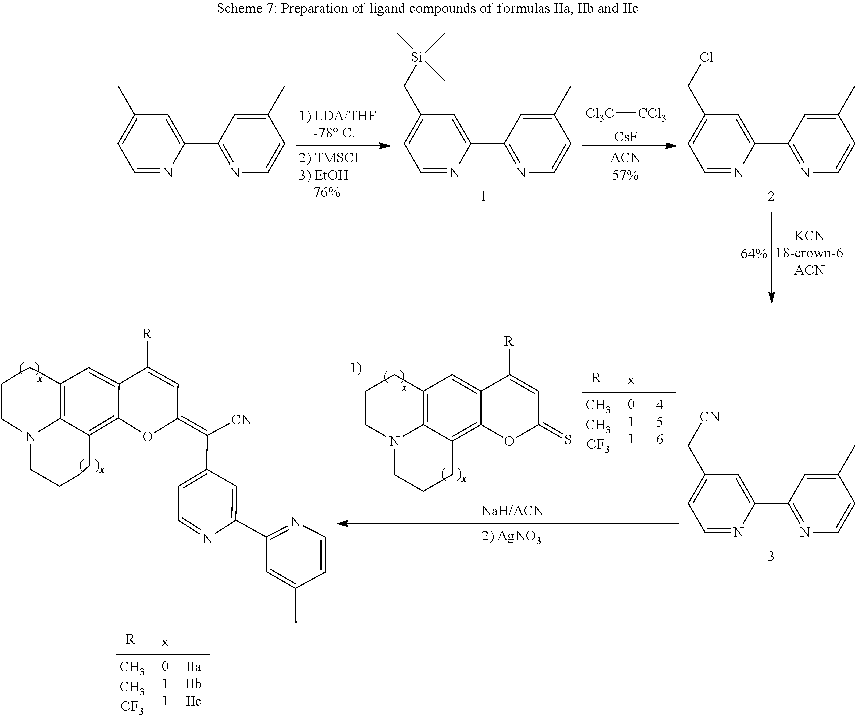

4-Methyl-4′-((trimethylsilyl)methyl)-2,2′-bipyridine, of formula 1. A solution of 4,4′-dimethyl-2,2′-bipyridine (2 g, 10.86 mmol) in anhydrous THF (80 mL) was added dropwise via cannula to a cold (−78° C.) solution of LDA in THF (12 mL, 11.95 mmol) under an Ar atmosphere. The resulting maroon mixture was stirred for 1 h at −78° C. Then, trimethylsilyl chloride (2 mL, 11.95 mmol) was added to the crude, which turned to blue and, exactly 10 seconds later, 10 mL of absolute ethanol were added carefully, which caused the solution to become yellow. The crude was transferred to a separatory funnel containing a saturated solution of NaHCO₃ (200 mL) and extracted with DCM (3×150 mL). The combined organic phases were washed with brine (150 mL), dried over anhydrous Na₂SO₄, filtered and evaporated to dryness to obtain 2.12 g of the title compound, as a yellow solid (yield: 76%), which was used without further purification.

4-(Chloromethyl)-4′-methyl-2,2′-bipyridine, of formula 2. 4-Methyl-4′-((trimethylsilyl)methyl)-2,2′-bipyridine (2.12 g, 7.80 mmol), hexachloroethane (7.39 g, 31.2 mmol) and cesium fluoride (4.76 g, 31.2 mmol) were dissolved in anhydrous ACN (120 mL) under an Ar atmosphere and the resulting solution was stirred at 60° C. for 3.5 h. The reaction mixture was partitioned between 50 mL of H₂O and 50 mL of AcOEt and transferred to a separatory funnel. The aqueous phase was extracted with ethyl acetate (3×50 mL) and the combined organic phases were washed with brine (50 mL), dried over anhydrous Na₂SO₄, filtered and evaporated to dryness. The product was purified by column chromatography (silica gel, 0-10% MeOH in DCM) to obtain 1.01 g of the title compound, as a yellow solid (yield: 57%). TLC: Rf (1:9 MeOH/DCM) 0.6. LR-ESI MS (ESI): m/z 218.8 calc. for [C₁₂H₁₁N₂Cl+H]⁺: 218.1.

2-(4′-Methyl-[2,2′-bipyridin]-4-yl) acetonitrile, of formula 3. 4-(Chloromethyl)-4′-methyl-2,2′-bipyridine (1 g, 4.57 mmol), 18-crown-6 (26.8 mg, 0.09 mmol) and KCN (3.39 g, 36.5 mmol) were dissolved in 100 mL of ACN and stirred overnight at room temperature. HPLC-MS analysis of the crude revealed that the product was barely formed. Then, 214 mg of 18-crown-6 (0.81 mmol) were added and the solution was stirred overnight at 50° C. After confirmation by HPLC-MS that the reaction had finished, the reaction mixture was evaporated to dryness, re-dissolved in deionized water (100 mL) and extracted with DCM (3×100 mL). The combined organic phases were dried over anhydrous Na₂SO₄, filtered and evaporated to dryness. After purification by column chromatography (silica gel, 50-100% AcOEt in hexanes), 615 mg of the title compound were obtained as a white solid (yield: 64%). TLC: Rf (AcOEt 100%) 0.7. HR-ESI MS (ESI): m/z 210.1026 calc. for [C₁₃H₁₁N₃+H]+: 210.1025.

Thiocoumarin of formula 4. 7-Diethylamino-4-methylcoumarin (1.72 g, 7.42 mmol) and Lawesson's reagent (1.65 g, 4.08 mmol) were dissolved in toluene (40 mL) and heated at 100° C. for 12 h. After evaporation under reduced pressure, the dark residue was purified by column chromatography (silica gel, DCM) to give the thiocoumarin of formula 4, as an orange solid (1.70 g, 92%): TLC Rf (DCM) 0.50. HRMS (ESI-TOF) m/z [M+H]⁺ calcd. for C₁₄H₁₈NOS 248.1109, found 248.1099.

Thiocoumarin of formula 5. 2,3,6,7-Tetrahydro-9-methyl-1H,5H-quinolizino[9,1-gh]coumarin (2 g, 7.8 mmol) and Lawesson's reagent (1.89 g, 4.7 mmol) were dissolved in 100 mL of toluene and stirred overnight at 100° C. The dark-green crude was evaporated to dryness and the product was isolated by column chromatography (silica gel, 0-70% DCM in hexanes). 1.44 g of the thiocoumarin of formula 5 as an orange solid were obtained (yield: 68%). TLC: Rf (100% DCM) 0.8. HR-ESI MS (ESI): m/z 272.1104 calc. for [C₁₆H₁₇NOS+H]⁺: 272.1102.

Thiocoumarin of formula 6. 2,3,6,7-Tetrahydro-9-trifluoromethyl-1H,5H-quinolizino[9,1-gh]coumarin (1 g, 3.23 mmol) and Lawesson's reagent (791 mg, 1.94 mmol) were dissolved in toluene (50 mL). The yellow solution was stirred overnight at 100° C. The dark-red solution was evaporated to dryness and the thiocoumarin of formula 6 was isolated through column chromatography (silica-gel, 0-40% DCM in hexanes). 1.14 g of a golden maroon solid were obtained (yield: 95%). Golden maroon solid. TLC: Rf (100% DCM) 0.9. HR-ESI MS (ESI): m/z 326.0821 calc. for [C₁₆H₁₄F₃NOS+H]⁺: 326.0816.

Ligand compound of formula Ila. To a solution of sodium hydride (568 mg of a 60% dispersion in mineral oil, 14.15 mmol) and compound 3 (712 mg, 3.40 mmol) in anhydrous acetonitrile (300 mL), 7-(N,N-diethylamino-4-methyl-2-thiocoumarin (703 mg, 5.66 mmol) was added. The orange solution was stirred for 3 h. Then, silver nitrate (962 mg, 4.98 mmol) was added and the reaction mixture was stirred for 2 h at room temperature. The dark-maroon solution was analyzed by HPLC-MS to confirm the formation of the desired product in the crude, which was evaporated under reduced pressure. The product was isolated by silica column chromatography starting with hexanes and increasing the eluent polarity with DCM first (0-100%) and then with MeOH (0-10%). 451 mg of an orange/red solid were obtained (yield: 37%), identified as ligand compound of formula Ila. TLC: Rf (1:9 MeOH/DCM) 0.5. HR-ESI MS (ESI): m/z 423.2181 calc. for [C₂₇H₂₆N₄O+H]⁺: 423.2179

Ligand compound of formula IIb. To a solution of NaH (38 mg of a 60% dispersion in mineral oil, 1.58 mmol) and compound 3 (46 mg, 0.22 mmol) in anhydrous ACN (12 mL), thiocoumarin of formula 5 (50 mg, 0.18 mmol) was added. The maroon solution was stirred at 50° C. for 3 h. Then, silver nitrate (71 mg, 0.41 mmol) was added and the crude was stirred for 2 h at room temperature. The product was isolated by silica column chromatography starting with hexanes and increasing the eluent polarity with DCM first (0-100%) and then with MeOH (0-10%). 20 mg of an orange/brown solid were obtained (yield: 20%), identified as ligand compound of formula IIb. TLC: Rf (5% MeOH/DCM) 0.5. HR-ESI MS (ESI): m/z 447.2186 calc. for [C₂₉H₂₆N₄O+H]⁺: 447.2179.

Ligand compound of formula IIc. To a solution of NaH (18 mg of a 60% dispersion in mineral oil, 0.77 mmol) and compound 3 (41 mg, 0.18 mmol) in anhydrous ACN (35 mL), thiocoumarin of formula 6 (50 mg, 0.15 mmol) was added. The maroon solution was stirred for 3 h. Then, silver nitrate (56 mg, 0.32 mmol) was added and the crude was stirred for 2 hat room temperature. The product was isolated by silica column chromatography starting with hexanes and increasing the eluent polarity with DCM first (0-100%) and then with MeOH (0-20%). 58 mg of bright red solid were obtained (yield: 75%), identified as ligand compound of formula IIc. Bright red solid. TLC: Rf (5% MeOH/DCM) 0.5. HR-ESI MS (ESI): m/z 501.1889 calc. for [C₂₉H₂₃F₃N₄O+H]⁺: 501.1887.

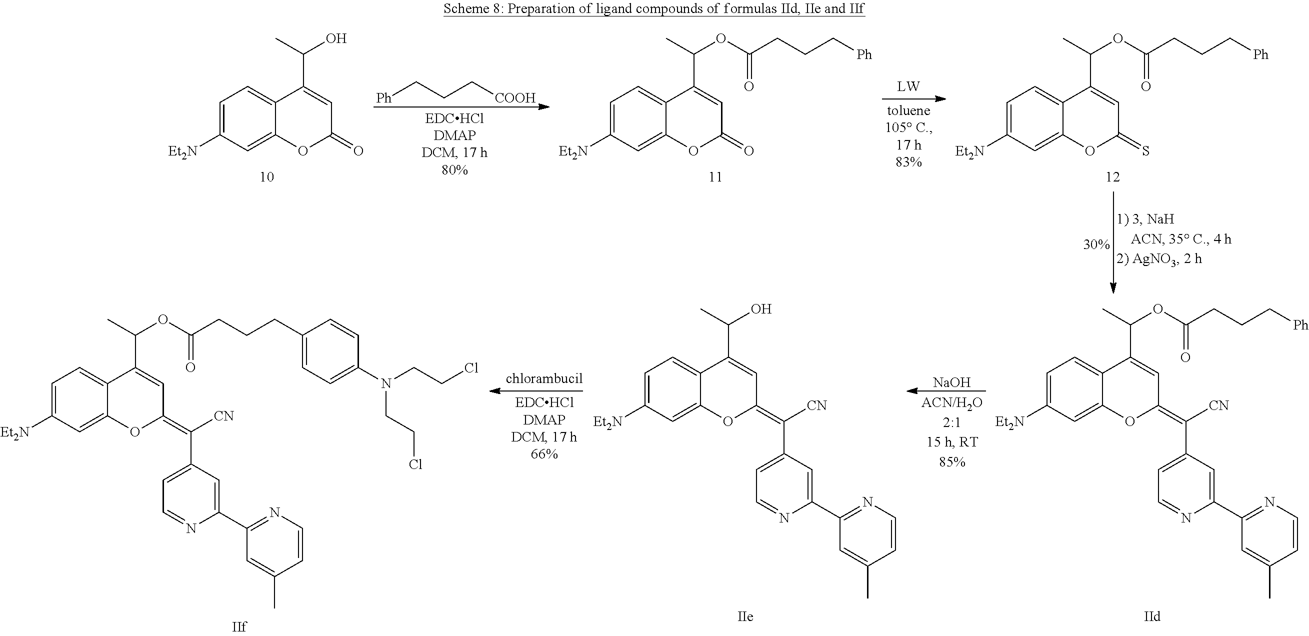

Compound of formula 11. A mixture of the coumarin of formula 10 (1.60 g, 6.12 mmol), phenylbutyric acid (1.50 g, 9.15 mmol), EDC (1.75 g, 9.15 mmol) and 4-dimethylaminopyridine (DMAP) (1.12 g, 9.15 mmol) was cooled at 0° C. under an argon atmosphere and then dissolved in DCM (100 mL). The mixture was stirred at 0° C. for 15 minutes and then 17 h at room temperature. Then, the solution was washed with saturated NH₄Cl (2×100 mL), 5% aqueous NaHCO₃ (1×100 mL, 2×50 mL) and deionized water (100 mL). The organic layer was dried over anhydrous MgSO₄, filtered and evaporated under reduced pressure. The product was isolated by silica column chromatography (silica gel, 50-100% DCM in hexanes, 1-3% MeOH in DCM) to give 2 g of the title compound as a yellow/orange solid (yield: 80%). TLC: Rf (DCM) 0.6. HR-ESI MS (ESI): m/z 408.2169 calc. for [C₂₅H₂₉NO₄+H]⁺: 408.2175.

Compound of formula 12. Lawesson's reagent (1.54 g, 3.81 mmol) was added to a solution of compound of formula 11 (1.94 g, 4.75 mmol) in toluene (60 mL) under an Ar atmosphere. The mixture was stirred at 105° C. in the dark overnight. A color change was observed from dark brown to pale yellow color. After removal of the solvent under reduced pressure, the product was isolated by column chromatography (silica gel, 100-30% hexanes in DCM) to give 1.67 g of the title compound as an orange solid (yield: 83%). TLC: R_{f} (DCM)=0.78. HR-ESI MS (ESI): m/z 424.1936 calc. for [C₂₅H₂₉NO₃S+H]⁺: 424.1946.

Ligand compound of formula IId. First, sodium hydride (68.3 mg of a 60% dispersion in mineral oil, 1.71 mmol) and 2-(4′-methyl-[2,2′-bipyridin]-4-yl)acetonitrile (formula 3) (357 mg, 1.71 mmol) were dissolved in anhydrous ACN (30 mL) and stirred for 15 min at 35° C. under an argon atmosphere. Then, a solution of the coumarin of formula 12 (362 mg, 0.85 mmol) in anhydrous ACN (15 mL) and added to the previous flask and the resulting reaction mixture was stirred for a further 4 h in the dark at 35° C. under an argon atmosphere. After 4 hours, AgNO₃ (363 mg, 2.14 mmol, 2.5 equiv.) was added and it was stirred for another 2 h. A color change was observed from brown to dark red/maroon color. After evaporation of the solvent under reduced pressure, the product was isolated by silica column chromatography starting with a 1:1 mixture of DCM and hexanes and increasing the eluent polarity with DCM by 10 until 100% and then with MeOH (0-1%) in DCM. 155 mg of an orange/red solid were obtained (yield: 30%), identified as the ligand compound of formula IId. TLC: R_{f} (10% MeOH in DCM)=0.4. HR-ESI MS (ESI): m/z 599.3014 calc. for [C₃₈H₃₈N₄O₃+H]⁺: 599.3022.

Ligand compound of formula Ile. To a solution of the ligand compound of formula IId (41.7 mg, 0.070 mmol) in 2:1 (v/v) ACN/H₂O (7 mL), 10% NaOH (6.65 mL, 0.070 mmol) was added and the reaction mixture was stirred overnight at room temperature in the dark. Reaction mixture color changed from orange/yellow to a bright orange with solid particles. After evaporation to dryness under reduced pressure, the crude was purified by flash chromatography (DCM in hexanes (50-100%) and then MeOH in DCM (0-10%), Puriflash system, Silica column PF-DLE-F0012) affording 26.9 mg of an orange/red solid (yield: 85%), identified as the ligand compound of formula Ile. TLC: Rf (10% MeOH in DCM) 0.57. HR-ESI MS (ESI): m/z 452.56 calc. for [C₂₉H₂₈N₄O₂+H]⁺: 453.2291.

Ligand compound of formula IIf. Chlorambucil (46.54 mg, 0.153 mmol), 4-dimethylaminopyridine (DMAP) (18.69 mg, 0.153 mmol) and 1-ethyl-3-(3′-dimethylaminopropyl)carbodiimide hydrochloride (EDC·HCl) (29.33 mg, 0.153 mmol) were added under argon atmosphere into a round bottom flask containing compound of formula 14 (46.10 mg, 0.102 mmol) which was places in an ice bath. The reagents were dissolved in DCM (13 mL) and the reaction mixture stirred for 15 min. Then, the flask was removed from the ice bath and allowed to react overnight in the dark at room temperature. Once the reaction had finished (confirmed by TLC and HPLC) the DCM was evaporated under reduced pressure and then purified via column chromatography (silica gel, DCM in hexane 0-100%, then MeOH in DCM 0-1%) to afford 49.9 mg of an orange/red solid (yield: 66%), identified as the ligand compound of formula IIf. TLC: Rf (10% MeOH in DCM) 0.75. HR-ESI MS (ESI): m/z 739.85 calc. for [C₄₂H₄₅Cl₂N₅O₃+H]⁺: 738.2978.

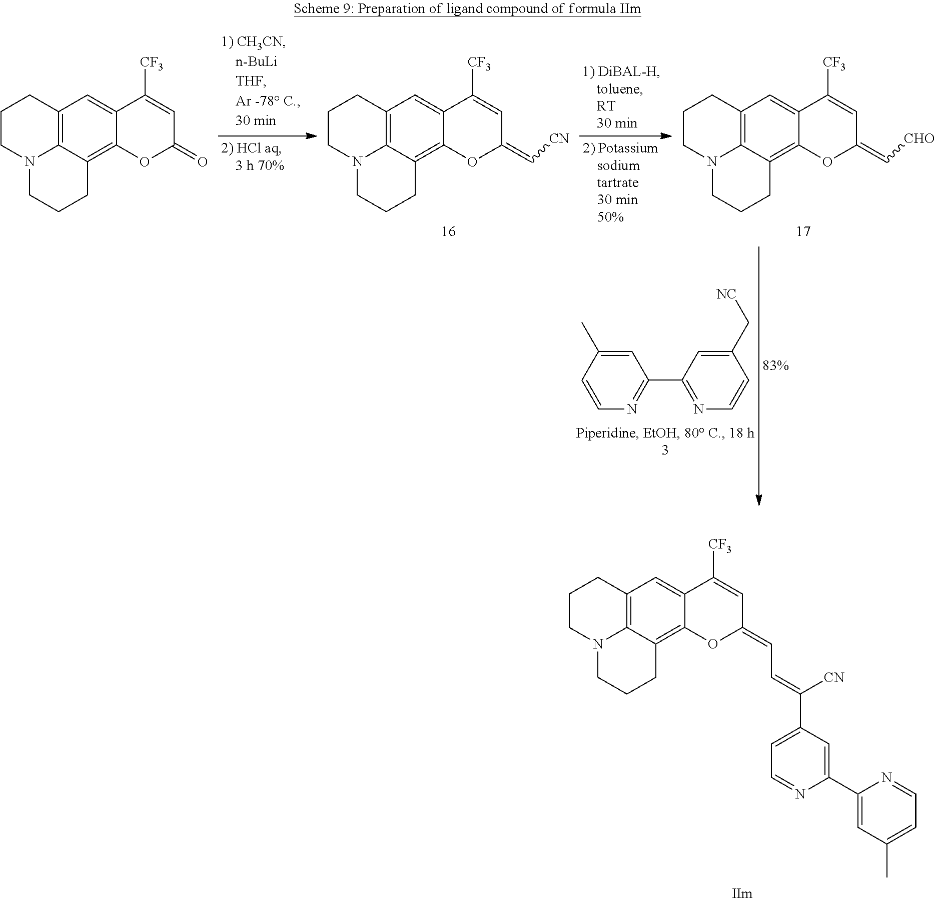 [SMILES: O=c1cc(C(F)(F)F)c2cc3c4c(c2o1)CCCN4CCC3 ; N#CC=C1C=C(C(F)(F)F)c2cc3c4c(c2O1)CCCN4CCC3 ; O=CC=C1C=C(C(F)(F)F)c2cc3c4c(c2O1)CCCN4CCC3 ; Cc1ccnc(-c2cc(CC#N)ccn2)c1 ; Cc1ccnc(-c2cc(/C(C#N)=C/C=C3/C=C(C(F)(F)F)c4cc5c6c(c4O3)CCCN6CCC5)ccn2)c1 ; "IIm"]

Nitrile coumarin of formula 16. To a cold solution (−78° C.) of CH₃CN (0.7 mL, 13.4 mmol) in anhydrous THF (40 mL), n-BuLi (5.2 mL, 2.5 M in hexanes, 13 mmol) was added under an Ar atmosphere. The resulting transparent and colorless solution was stirred for 15 min at −78° C., time after which the appearance of a white suspension was observed. Then, a solution of 2,3,6,7-tetrahydro-9-trifluoromethyl-1H,5H-quinolizino[9,1-gh] coumarin (1.023 g, 3.31 mmol) in anhydrous THF (35 mL) was slowly added and the reaction mixture was stirred for 30 min at −78° C. under Ar. The resulting orange mixture was quenched by addition of saturated aqueous NH₄Cl (35 mL) while still at −78° C., and a white solid precipitated. The mixture was warmed to room temperature and extracted with AcOEt (3×50 mL). The combined organic phases were evaporated to dryness. To the resulting crude oil, 0.5 M aqueous HCl (200 mL) was added and the mixture stirred vigorously for 3 h. The solution color turned from orange to brown. This solution was extracted with AcOEt (3×80 mL), dried over anhydrous MgSO₄, filtered and evaporated to dryness under reduced pressure. The crude mixture was purified by flash chromatography silica gel, 0-15% AcOEt in hexanes. 760 mg of the title compound, as bright orange solid were obtained (yield: 70%). Orange solid. TLC: Rf (25% AcOEt in hexanes) 0.38. HR-MS (ESI): m/z 332.1209 calc. for [C₁₈H₁₅F₃N₂O+H]⁺: 333.1215.

Coumarin aldehyde of formula 17. Nitrile coumarin of formula 16 (300 mg, 0.90 mmol) was dissolved in anhydrous toluene (35 mL) under an Ar atmosphere and the mixture was let stir for 10 min (orange solution). Then, 1 M toluene solution of DIBALH was added (1.35 mL, 1.35 mmol) and the reaction mixture stirred for 30 min at room temperature under an Ar atmosphere (the color changed to maroon). The crude was cooled in an ice-bath and acetone (4 mL) was added for decomposition of the excess reagent (the color changed from maroon to dark red). After addition of saturated potassium sodium tartrate (25 mL), the crude was extracted with AcOEt (3×25 mL) and the organic phases washed with water (25 mL) and brine (25 mL), dried over anhydrous MgSO4, filtered and evaporated to dryness under reduced pressure. The compound was purified by silica column chromatography with hexanes increasing the polarity with DCM first (0-100%) and with MeOH then (0-0.5%). 150 mg of the title compound, as a red solid, were obtained (yield: 50%). TLC: Rf (5% MeOH in DCM) 0.74. HR-MS (ESI): m/z 336.1206 calc. For [C₁₈H₁₆F₃NO₂+H]⁺: 336.1211.

Ligand compound of formula IIm. To a solution of the coumarin aldehyde of formula 17 (50 mg, 0.15 mmol) in absolute ethanol (7 mL), piperidine (70 mg, 0.83 mmol) and 2-(4′-methyl-[2,2′-bipyridin]-4-yl)acetonitrile (46 mg, 0.22 mmol) were added. The reaction mixture (dark maroon) was stirred at 80° C. overnight. After evaporation to dryness under reduced pressure, the compound was purified by silica column chromatography with hexanes increasing the polarity with DCM first (0-100%) and with MeOH then (0-2%). 66 mg of a violet solid, identified as the ligand compound of formula IIm, were obtained (yield: 83%). TLC: Rf (5% MeOH in DCM) 0.4. HR-ESI MS (ESI): m/z 527.2077 calc. for [C₃₁H₂₅F₃N₄O+H]⁺: 527.2053.

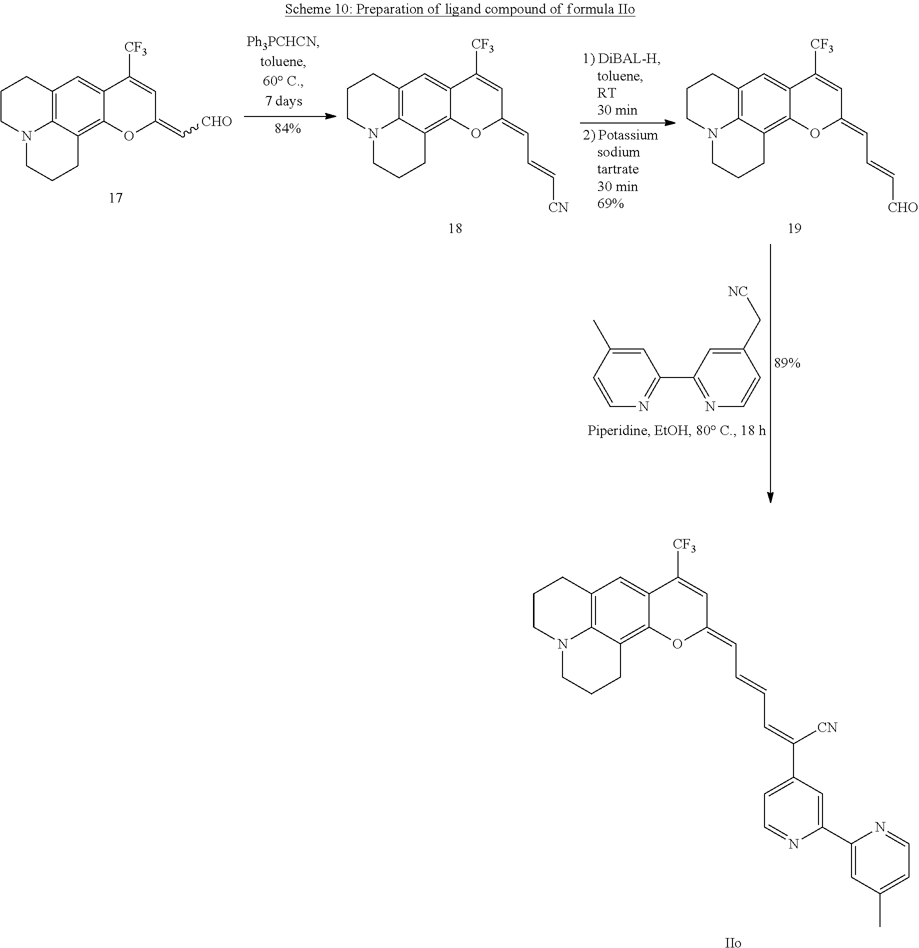 [SMILES: O=CC=C1C=C(C(F)(F)F)c2cc3c4c(c2O1)CCCN4CCC3 ; N#C/C=C/C=C1/C=C(C(F)(F)F)c2cc3c4c(c2O1)CCCN4CCC3 ; O=C/C=C/C=C1/C=C(C(F)(F)F)c2cc3c4c(c2O1)CCCN4CCC3 ; Cc1ccnc(-c2cc(CC#N)ccn2)c1 ; Cc1ccnc(-c2cc(/C(C#N)=C/C=C/C=C3/C=C(C(F)(F)F)c4cc5c6c(c4O3)CCCN6CCC5)ccn2)c1 ; "IIo"]

Nitrile coumarin of formula 18. To a solution of the coumarin aldehyde formula 17 (207 mg, 0.62 mmol) in toluene (15 mL), (triphenylphosphoranylidene)acetonitrile (850 mg, 2.82 mmol) was added. The solution was degassed with Ar and then the vial was sealed, and the mixture was stirred at 60° C. for 7 days. Once the starting aldehyde was totally consumed, deionized water (H₂O) was added to the crude and then extracted with AcOEt (3×30 mL). The combined organic phases were dried over anhydrous MgSO₄, filtered, and evaporated to dryness under reduced pressure and the crude was purified by silica column chromatography with hexanes increasing the polarity with AcOEt (0-12%). 184 mg of the title compound, as a red solid, were obtained (yield: 84%). TLC: Rf (50% AcOEt in hexanes) 0.90. HR-MS (ESI): m/z 359.1366 calc. for [C₂₀H₁₇F₃N₂O+H]⁺: 359.1357.

Coumarin aldehyde of formula 19. Nitrile coumarin of formula 18 (103 mg, 0.29 mmol) was dissolved in anhydrous THE (30 mL) under an Ar atmosphere and the mixture was cooled in an ice bath. Then, 1 M toluene solution of DIBALH was added (3.0 mL, 2.95 mmol) and the reaction mixture stirred for 30 min at room temperature under an Ar atmosphere. The crude was cooled in an ice-bath and acetone (10 mL) was added for decomposition of the excess reagent. After addition of saturated potassium sodium tartrate (25 mL) and let stir for 30 min, the crude was extracted with DCM (3×50 mL) and the organic phases washed with brine (50 mL), dried over anhydrous MgSO4, filtered and evaporated to dryness under reduced pressure. The compound was purified by silica column chromatography with hexanes increasing the polarity with DCM first (0-100%) and with MeOH then (0-0.5%). 78 mg of the title compound, as a yellow solid, were obtained (yield: 69%). TLC: Rf (5% MeOH in DCM) 0.52. HR-MS (ESI): m/z 362.1362 calc. for [C₂₀H₁₇F₃N₂O+H]⁺: 362.1359.

Ligand compound of formula IIo. To a solution of the coumarin aldehyde of formula 19 (32 mg, 0.088 mmol) in absolute ethanol (10 mL), piperidine (45 mg, 0.531 mmol) and 2-(4′-methyl-[2,2′-bipyridin]-4-yl)acetonitrile (23 mg, 0.11 mmol) were added. The reaction mixture was stirred at 80° C. overnight. After evaporation to dryness under reduced pressure, the compound was purified by silica column chromatography with hexanes increasing the polarity with DCM first (0-100%) and with MeOH then (0-1%). 44 mg of a violet solid, identified as the ligand compound of formula IIo, were obtained (yield: 89%). TLC: Rf (5% MeOH in DCM) 0.59. LR-MS (ESI): m/z 553.25 calc. for [C₃₃H₂₇F₃N₄O+H]⁺: 553.2210.

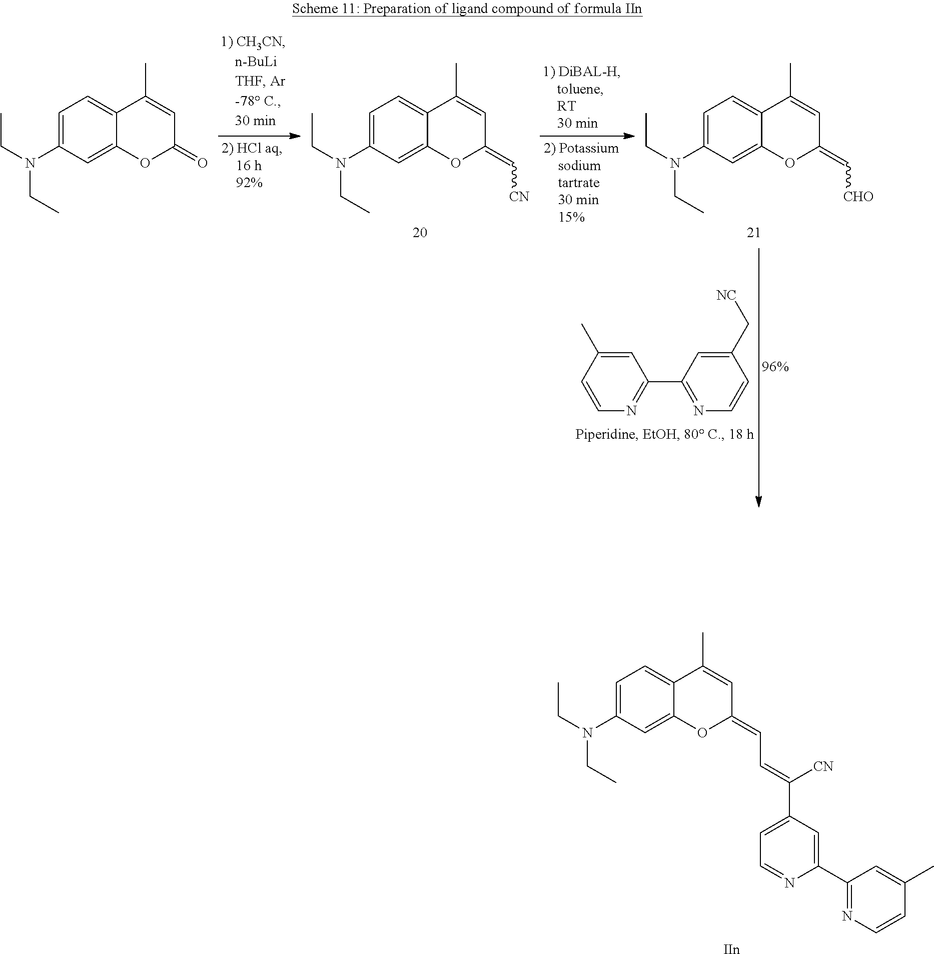 [SMILES: CCN(CC)c1ccc2c(C)cc(=O)oc2c1 ; CCN(CC)c1ccc2c(c1)OC(=CC#N)C=C2C ; CCN(CC)c1ccc2c(c1)OC(=CC=O)C=C2C ; Cc1ccnc(-c2cc(CC#N)ccn2)c1 ; CCN(CC)c1ccc2c(c1)O/C(=C\C=C(/C#N)c1ccnc(-c3cc(C)ccn3)c1)C=C2C ; "IIn"]

Nitrile coumarin of formula 20. To a cold solution (−78° C.) of CH₃CN (2.42 mL, 34.6 mmol) in anhydrous THF (40 mL), n-BuLi (13.8 mL, 2.5 M in hexanes, 34.6 mmol) was added under an Ar atmosphere. The resulting transparent and colorless solution was stirred for 15 min at −78° C., time after which the appearance of a white suspension was observed. Then, a solution of 7-(diethylamino)-4-methyl-2H-chromen-2-one coumarin (2.008 g, 8.65 mmol) in anhydrous THF (30 mL) was slowly added and the reaction mixture was stirred for 30 min at −78° C. under Ar. The resulting white mixture was quenched by addition of saturated aqueous NH₄Cl (35 mL) while still at −78° C., and a white solid precipitated. The mixture was warmed to room temperature and extracted with AcOEt (3×50 mL). The combined organic phases were evaporated to dryness. To the resulting orange crude oil, 0.5 M aqueous HCl (200 mL) was added and the mixture stirred vigorously for 16 h. The solution turned into brown color and a yellow solid precipitated. The precipitate was dissolved adding 50 mL of DCM and the mixture was extracted with DCM (3×100 mL), dried over anhydrous MgSO₄, filtered and evaporated to dryness under reduced pressure to obtain a brown solid. The crude mixture was purified by silica column chromatography (0-22% AcOEt in hexanes). 2.01 g of the title compound, as bright orange solid were obtained (yield: 92%). Orange solid. TLC: Rf (50% AcOEt in hexanes) 0.61. LR-ESI MS (ESI): m/z 255.2 calc. for [C₁₆H₁₈N₂O+H]⁺: 255.1492.

Coumarin aldehyde of formula 21. Nitrile coumarin of formula 20 (500 mg, 1.97 mmol) was dissolved in anhydrous toluene (25 mL) under an Ar atmosphere and the mixture was let stir for 10 min (orange solution). Then, 1 M toluene solution of DIBALH was added (3.0 mL, 2.95 mmol) and the reaction mixture stirred for 30 min at room temperature under an Ar atmosphere. The crude was cooled in an ice-bath and acetone (10 mL) was added for decomposition of the excess reagent. After addition of saturated potassium sodium tartrate (40 mL), the crude was extracted with DCM (3×50 mL) and the organic phases washed with brine (50 mL), dried over anhydrous MgSO4, filtered and evaporated to dryness under reduced pressure. The compound was purified by silica column chromatography with hexanes increasing the polarity with DCM first (0-100%) and with MeOH then (0-0.5%). 78 mg of the title compound, as a yellow solid, were obtained (yield: 15%). TLC: Rf (5% MeOH in DCM) 0.52. LR-ESI MS (ESI): m/z 258.2 calc. for [C₁₆H₁₉NO₂+H]⁺: 258.1489.

Ligand compound of formula IIn. To a solution of the coumarin aldehyde of formula 21 (47 mg, 0.18 mmol) in absolute ethanol (10 mL), piperidine (84 mg, 1.0 mmol) and 2-(4′-methyl-[2,2′-bipyridin]-4-yl)acetonitrile (44 mg, 0.20 mmol) were added. The reaction mixture was stirred at 80° C. overnight. After evaporation to dryness under reduced pressure, the compound was purified by silica column chromatography with hexanes increasing the polarity with DCM first (0-100%) and with MeOH then (0-2%). 79 mg of a violet solid, identified as the ligand compound of formula IIn, were obtained (yield: 96%). TLC: Rf (5% MeOH in DCM) 0.38. HR-ESI MS (ESI): m/z 449.2336 calc. for [C₂₉H₂₈N₄O+H]⁺: 449.2336.

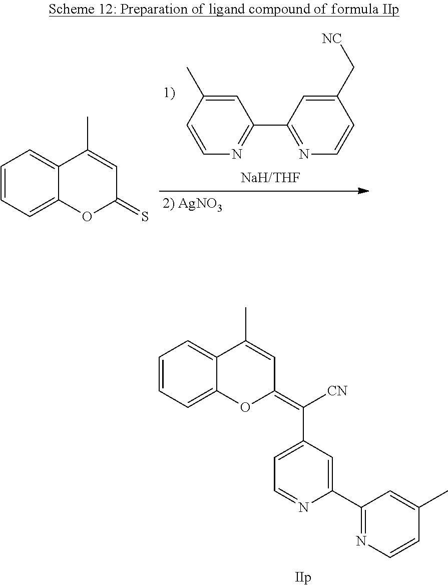 [SMILES: Cc1cc(=S)oc2ccccc12 ; Cc1ccnc(-c2cc(CC#N)ccn2)c1 ; CC1=C/C(=C(\C#N)c2ccnc(-c3cc(C)ccn3)c2)Oc2ccccc21]

Ligand compound of formula IIp. First, sodium hydride (66 mg of a 60% dispersion in mineral oil, 1.64 mmol) and 2-(4′-methyl-[2,2′-bipyridin]-4-yl)acetonitrile (106 mg, 0.506 mmol) were dissolved in anhydrous THF (15 mL) and stirred for 15 min at 40° C. under an Ar atmosphere. Then, a solution of 4-methyl-2H-chromene-2-thione (85 mg, 0.482 mmol) in anhydrous THF (15 mL) and added to the previous flask and the resulting reaction mixture was stirred for a further 4 h in the dark at 40° C. under an Ar atmosphere. After 4 h, AgNO₃ (163 mg, 0.964 mmol) was added and it was stirred for another 2 h at 35° C. After evaporation of the solvent under reduced pressure, the compound was purified by silica column chromatography with hexanes increasing the polarity with DCM first (0-100%) and with MeOH then (0-1%). 92 mg of an orange solid were obtained (yield: 54%), identified as the ligand compound of formula IIp. TLC: Rf (2.5% MeOH in DCM) 0.25. HR-MS (ESI): m/z 352.1451 calc. for [C₂₃H₁₇N₃O+H]+: 352.1444.

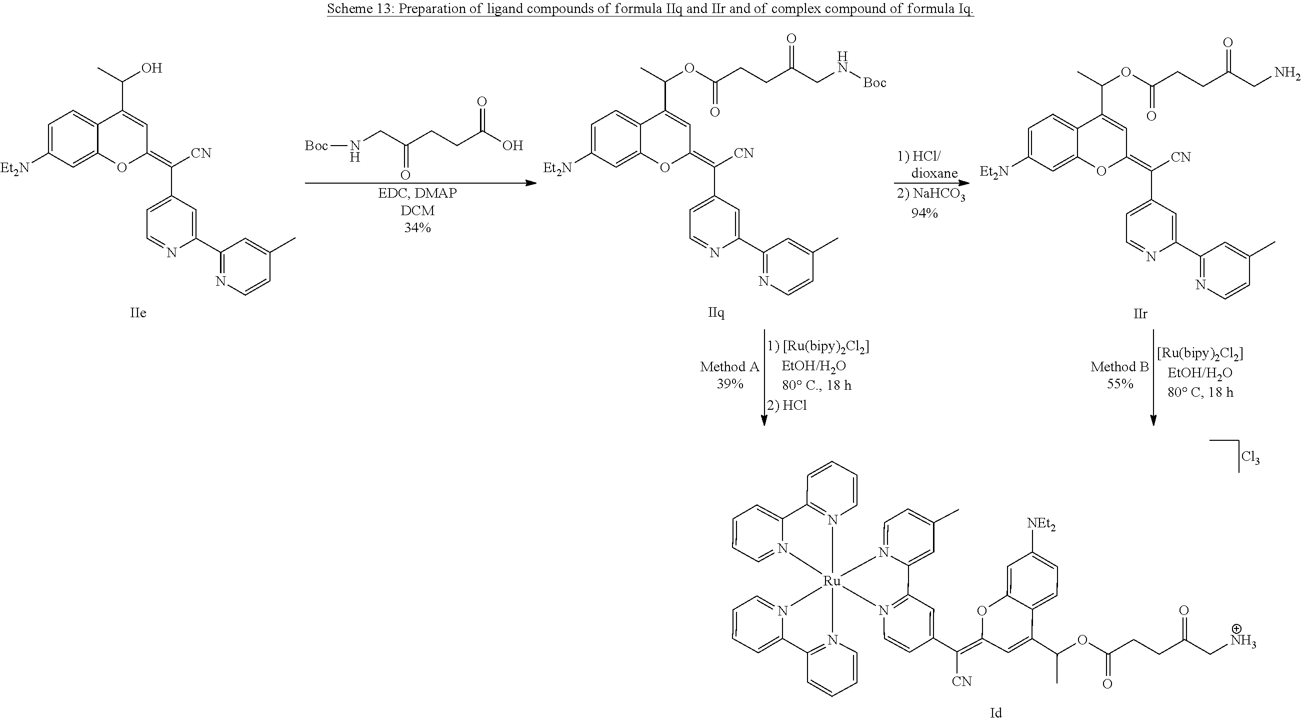

Ligand compound of formula 11g. 4-Dimethylaminopyridine (DMAP) (20 mg, 0.164 mmol) and 1-ethyl-3-(3′-dimethylaminopropyl)carbodiimide hydrochloride (EDC-HCl) (38 mg, 0.198 mmol) were sequentially added to an ice-cooled solution of N-Boc-5-aminolevulinic acid (38 mg, 0.164 mmol) in DCM (10 mL) under an argon atmosphere. After the mixture was stirred for 15 min at 0° C., a solution of compound of formula Ile (61 mg, 0.135 mmol) in DCM (5 mL) was added dropwise at the same temperature. Then, the ice bath was removed, and the resulting mixture was further stirred for 16 h at room temperature. After completion of the reaction (TLC, HPLC), the volatiles were concentrated under reduced pressure and the resulting crude was purified by flash column chromoatography (silica gel, MeOH in DCM 0-5%) to afford 31 mg of an orange/red solid (yield: 34%), identified as the ligand compound of formula IIq. LR-MS (ESI): m/z [M+H]⁺ calcd for C₃₈H₄₃N₅O₆ 666.33, found 666.57.

Ligand compound of formula IIr. To an ice-cooled solution of ligand compound of formula IIq (15 mg, 0.022 mmol) in anhydrous THF (2 mL) was added 4N HCl in dioxane (675 μL, 2.70 mmol), and the resulting mixture was allowed to warm to room temperature and further stirred for 5 h. After completion of the reaction (HPLC-MS), the reaction mixture was neutralized with 10% (w/v) aq. NaHCO₃ (10 mL), diluted with water (20 mL), extracted with DCM (3×20 mL), and the combined organic extracts were dried over anhydrous MgSO₄, filtered and concentrated under reduced pressure to give the crude ligand compound of formula IIr (12 mg, yield: 94%) as an orange/red solid, which was used in the following step without any further purification. LR-MS (ESI): m/z [M+H]⁺ calcd for C₃₃H₃₅N₅O₄ 566.28, found 566.45.

Complex compound of formula 1g. Method A: Ligand compound of formula IIq (16 mg, 0.024 mmol) and [Ru(bpy)₂Cl₂] (10 mg, 0.021 mmol) were dissolved in 4 mL of a 3:1 (v/v) solution of EtOH/H₂O under an Ar atmosphere. The reaction mixture was stirred overnight at 80° C. and analyzed by HPLC-MS to confirm the formation of the product. The reaction mixture was evaporated to dryness and the residue was taken up in anhydrous THF (2 mL), treated with 4N HCl in dioxane (600 μL, 2.37 mmol) at 0° C., allowed to warm to room temperature and further stirred for 5 h. After completion of the reaction (HPLC-MS), the reaction mixture was neutralized with 10% (w/v) aq. NaHCO₃ (5 mL), diluted with water (20 mL), extracted with DCM (3×20 mL), and the combined organic extracts were dried over anhydrous MgSO₄, filtered and concentrated under reduced pressure to give the crude. Method B: Ligand compound of formula IIr (11 mg, 0.019 mmol) and [Ru(bpy)₂Cl₂] (9 mg, 0.019 mmol) were dissolved in 4 mL of a 3:1 (v/v) solution of EtOH/H₂O under an Ar atmosphere. The reaction mixture was stirred overnight at 80° C. and analyzed by HPLC-MS to confirm the formation of the product. The reaction mixture was evaporated to dryness to give the crude. In both methods, the crude was purified by preparative HPLC (C18 column; mobile phase, A: 0.1% HCOOH in H₂O, B: 0.1% HCOOH in ACN; gradient: 10 to 80% B in 30 min) and the desired complex compound of formula Iq was obtained as a maroon solid (Method A: 8 mg, yield: 39%; Method B: 10 mg, yield: 55%). Analytical HPLC (C18 column; mobile phase, A: 0.1% HCOOH in H₂O, B: 0.1% HCOOH in ACN; gradient: 5 to 100% B in 5 min): Rt=2.14 min. LR-MS (ESI): m/z [M]²⁺ calcd for C₅₃H₅₁NgO₄Ru²⁺ 489.65, found 489.60.

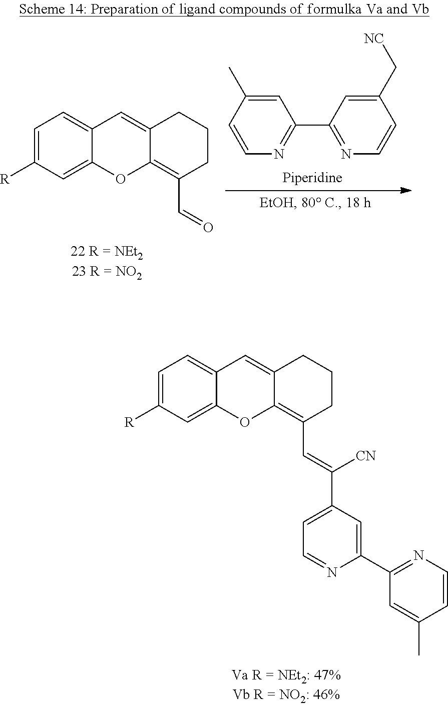

Ligand compound of formula Va. To a solution of the coumarin aldehyde of formula 22 (92 mg, 0.32 mmol) in absolute ethanol (10 mL), piperidine (166 mg, 2.0 mmol) and 2-(4′-methyl-[2,2′-bipyridin]-4-yl)acetonitrile (68 mg, 0.32 mmol) were added. The reaction mixture was stirred at 80° C. overnight. After evaporation to dryness under reduced pressure, the compound was purified by silica column chromatography with hexanes increasing the polarity with DCM first (0-100%) and with MeOH then (0-1%). 73 mg of a violet solid, identified as the ligand compound of formula Va, were obtained (yield: 47%). TLC: Rf (5% MeOH in DCM) 0.59. HR-MS (ESI): m/z 475.2495 calc. for [C₃₁H₃₀N₄O+H]⁺: 475.2492.

Ligand compound of formula Vb. To a solution of the coumarin aldehyde of formula 23 (40 mg, 0.155 mmol) in absolute ethanol (8 mL), piperidine (79 mg, 0.932 mmol) and 2-(4′-methyl-[2,2′-bipyridin]-4-yl)acetonitrile (33 mg, 0.155 mmol) were added. The reaction mixture was stirred at 80° C. overnight. After evaporation to dryness under reduced pressure, the compound was purified by silica column chromatography with hexanes increasing the polarity with DCM first (0-100%) and with MeOH then (0-2%). 32 mg of a brown solid, identified as the ligand compound of formula Vb, were obtained (yield: 46%). TLC: Rf (5% MeOH in DCM) 0.30. LR-MS (ESI): m/z 449.07 calc. for [C₂₇H₂₀N₄O₃+H]⁺: 449.1608.

### General Procedure for the Synthesis of Complex Compounds of Formulae in, Lo, Ip, IVa and IVb.

The corresponding ligand compound of formula IIn, IIo, IIp, Va or Vb and [Ru(bpy)₂Cl₂] (1.1 eq) were dissolved in a 1:1 (v/v) mixture of EtOH/H₂O and the reaction mixture was stirred for 24-48 h at 80-90° C. under an argon atmosphere. The reaction was followed by HPLC-MS. Once the total conversion was reached, the solvent was evaporated to dryness and the product was purified by silica column chromatography with DCM and increasing the eluent polarity with MeOH. Compounds were obtained as a racemic mixture of the chloride salt and characterized by HR-ESI MS.

In: HR-ESI MS (ESI): m/z 431.1336 calc. for [C₄₉H₄₄N₈ORu]²⁺: 431.1336. Analytical HPLC (5 to 100% B in 5 min, formic acid additive): Rt=2.46 min.

Io: HR-ESI MS (ESI): m/z 483.1282 calc. for [C₅₃H₄₃F₃N₈ORu]²⁺: 483.1272. Analytical HPLC (5 to 100% B in 5 min, formic acid additive): Rt=2.88 min.

Ip: HR-ESI MS (ESI): m/z 382.5898 calc. for [C₄₃H₃₃N₇ORu]²⁺: 382.5890. Analytical HPLC (5 to 100% B in 5 min, formic acid additive): Rt=2.25 min.

IVa: HR-ESI MS (ESI): m/z 444.1420 calc. for [C₅₁H₄₄N₈ORu]²⁺: 444.1414. Analytical HPLC (5 to 100% B in 5 min, formic acid additive): Rt=2.68 min.

IVb: HR-ESI MS (ESI): m/z 431.0972 calc. for [C₄₇H₃₆N₈O₃Ru]²⁺: 431.0972. Analytical HPLC (5 to 100% B in 5 min, formic acid additive): Rt=2.39 min.

### Phototoxicity Evaluation of Complex Compounds of Formula I or IV

The phototoxicity of several complex compounds of formula I or IV was assessed under normoxia (21% O₂) and hypoxia (2% O₂) conditions, towards several (human and mouse) cancer cell lines, and towards human normal cells. Obtained results are shown in accompanying Tables 1-7. In all cases, the phototoxic index (PI) was calculated as: PI=IC₅₀ (dark-non-irradiated cells)/IC₅₀ (irradiated cells).

Cell lines and cell culture conditions. The following cancer cell lines were used: CT-26 (mouse, colon carcinoma); HT-29 (human, colorectal adenocarcinoma); A-549 (human, lung adenocarcinoma); A-2780 (human, ovarian carcinoma). The following human normal cells were used: RPE-1 (human retinal pigment epithelial-1), HEK-293 (human embryonic kidney) and MRC-5 (human fetal lung fibroblast cells). Cell lines were cultured in their specific media and maintained in a humidified atmosphere at 37° C. with 5% CO₂. The CT-26 cell line was cultured in DMEM media (Gibco) supplemented with 10% fetal calf serum (Gibco) and 1% Penicillin-Streptomycin antibiotic (Gibco). The HT-29 cell line was cultured in McCoy media (Gibco) supplemented with 10% fetal calf serum (Gibco) and 1% Penicillin-Streptomycin antibiotic (Gibco). The A-549 cell line was cultured in DMEM/F-12 media (Gibco) supplemented with 10% fetal calf serum (Gibco) and 1% Penicillin-Streptomycin antibiotic (Gibco). The A-2780 cell line was cultured in RPMI media (Gibco) supplemented with 10% fetal calf serum and 1% Penicillin-Streptomycin antibiotic (Gibco). The RPE-1 cell line was cultured in DMEM/F-12 media (Gibco) supplemented with 10% fetal calf serum and 1% Penicillin-Streptomycin antibiotic (Gibco). The HEK-293 cell line was cultured in DMEM media (Gibco) supplemented with 10% fetal calf serum and 1% Penicillin-Streptomycin antibiotic (Gibco). The MRC-5 cell line was cultured in DMEM/F-12 media (Gibco) supplemented with 10% fetal calf serum and 1% Penicillin-Streptomycin antibiotic (Gibco).

Phototoxicity evaluation of complex compounds of formula I or IV, in 2D monolayer cells under normoxia (21% O₂). The phototoxicity of tested compounds under normoxia (21% O₂) was assessed by a fluorometric cell viability assay using Resazurin (Acros Organics). Briefly, cells were seeded in triplicate in 96-well plates at a density of 4×10³ cells/well in a final volume of 100 μL per well. After 24 h, cells were treated with increasing concentrations of the metal complexes and control compounds. Serial dilutions of the tested compounds with cell media from 10 mM or 25 mM stock solutions in DMSO were added at the final concentrations in the range of 0.001 to 250 μM in a final volume of 100 μL per well. The cells were incubated with the samples in the dark for 4 h and, after this time, the medium was refreshed. To study the phototoxic effect of the tested compounds, the cells were exposed to 540 nm (spectral half-width: 32 nm, 40 min, 3.75 mW cm⁻², 9.0 J cm⁻²), 645 nm (spectral half-width: 32 nm, 60 min, 2.50 mW cm⁻², 9.0 J cm⁻²), 670 nm (spectral half-width: 32 nm, 60 min, 3.75 mW cm⁻², 13.5 J cm⁻²), 740 nm (spectral half-width: 32 nm, 60 min, 3.50 mW cm⁻², 12.6 J cm⁻²) and 770 nm (spectral half-width: 32 nm, 60 min, 6.75 mV cm⁻² 24.3 J cm⁻²) light using an Atlas Photonics LUMOS BIO irradiator. To study the dark cytotoxicity of the samples, the cells were not irradiated. Then, 44 h treatment-free cell recovery period was allowed while maintaining the temperature throughout the experiment at 37° C. After 44 h of incubation, the medium was removed by suction, and 100 μL of complete medium containing Resazurin (0.2 mg/mL final concentration) was added. After 4 h of incubation at 37° C., the fluorescence signal of the Resorufin product was measured (λₑₓ=540 nm, λₑₘ=590 nm) using an Infinite 200 PRO Microplate Reader from TECAN. IC₅₀ values were then calculated based on the inhibitory rate curves. All experiments were performed in three independent studies with triplicate points per concentration level (n=3 biologically independent replicates).

Phototoxicity evaluation of complex compounds of formula I or IV, in 2D monolayer cells under hypoxia (2% O₂). The phototoxicity of the tested compounds under hypoxia (2% O₂) was assessed by a fluorometric cell viability assay using Resozurin (Acros Organics). Cells were seeded in triplicate in 96-well plates at a density of 2.5×10³ cells/well in a final volume of 100 μL per well in the incubator at 2% O₂. A flask containing the corresponding cell culture medium was also prepared in the incubator at 2% O₂. After standing for 48 h at low oxygen, cells were treated with increasing concentrations of the metal complexes and control compounds. Serial dilutions of the tested compounds with media from 10 mM or 25 mM stock solutions in DMSO were added at the final concentrations in the range of 0.01 to 250 μM in a final volume of 100 μL per well. The cells were incubated with the samples in the dark for 4 h and, after this time, the medium was refreshed. To study the phototoxic effect of the samples under hypoxia conditions, the hypoxia chamber Glove Box from Plas-Labs (856-Series) was used. The cells were exposed to light (see previous paragraph) using an Atlas Photonics LUMOS BIO irradiator which placed inside of the hypoxia chamber. To assess the dark cytotoxicity of the samples, cells were not irradiated. Then, a 44 h treatment-free cell recovery period inside the incubator at 2% 02 was allowed, and the temperature throughout the experiment was maintained at 37° C. After 44 h of incubation, the medium was removed by suction, and 100 μL of complete medium containing Resazurin (0.2 mg/mL final concentration) was added. After 4 h of incubation at 37° C. in the incubator at 21% 02, the fluorescence signal of the Resorufin product was measured (λₑₓ=540 nm, λₑₘ=590 nm) using an Infinite 200 PRO Microplate Reader from TECAN. IC₅₀ values were calculated as indicated in previous paragraph.

Phototoxicity results towards cancer cells of several complex compounds of formula I or IV, after irradiation with light of different wavelength (X), under normoxia and hypoxia conditions. Obtained values of IC₅₀ and phototoxic index (PI) in different conditions are shown in accompanying Tables 1-8. Uncertainty is standard error of the mean for n=3. “nd” means “non determined”.

TABLE 1
Phototoxicity of complex compounds of formulas Ia and Ic, towards CT-26
cancer cells, under normoxic (21% O₂) and hypoxic (2% O₂) conditions.

| | Complex compound of formula Ia | | | | Complex compound of formula Ic | | | |
|---|---|---|---|---|---|---|---|---|
| λ | Normoxia (21% O₂) | | Hypoxia (2% O₂) | | Normoxia (21% O₂) | | Hypoxia (2% O₂) | |
| (nm) | IC₅₀ (nM) | PI | IC₅₀ (nM) | PI | IC₅₀ (nM) | PI | IC₅₀ (nM) | PI |
| dark | >2.5 × 10⁵ | — | >2.5 × 10⁵ | — | >2.5 × 10⁵ | — | >2.5 × 10⁵ | — |
| 540 | 8.2 ± 0.6 | >30487 | 35 ± 5 | >7143 | 25 ± 2 | >104 | 86 ± 11 | >2907 |
| 645 | 48 ± 3 | >5208 | 920 ± 90 | >272 | 7.4 ± 0.6 | >33783 | 76 ± 8 | >3290 |
| 670 | 1460 ± 450 | >171 | 13240 ± 3640 | >19 | 36 ± 3 | >6944 | 74 ± 5 | >3378 |
| 740 | 31300 ± 6100 | >8 | >10⁵ | — | 760 ± 60 | >329 | 9560 ± 2150 | >26 |

TABLE 2
Phototoxicity of complex compounds of formulas Ig and Ih, towards CT-26
cancer cells, under normoxic (21% O₂) and hypoxic (2% O₂) conditions.

| | Complex compound of formula Ig | | | | Complex compound of formula Ih | | | |
|---|---|---|---|---|---|---|---|---|
| λ | Normoxia (21% O₂) | | Hypoxia (2% O₂) | | Normoxia (21% O₂) | | Hypoxia (2% O₂) | |
| (nm) | IC₅₀ (nM) | PI | IC₅₀ (nM) | PI | IC₅₀ (nM) | PI | IC₅₀ (nM) | PI |
| Dark | 9600 ± 2700 | — | 13300 ± 1300 | — | 10900 ± 2300 | — | 8300 ± 2000 | — |
| 645 | 64 ± 3 | 150 | 380 ± 90 | 35 | 43 ± 1 | 253 | 100 ± 8 | 83 |
| 670 | 290 ± 10 | 33 | 1070 ± 3640 | 13 | 36 ± 2 | 303 | 70 ± 5 | 118 |
| 740 | 2900 ± 1100 | 3 | nd | — | 220 ± 63 | 50 | nd | — |

TABLE 3
Phototoxicity of complex compound of formula Id towards
CT-26 cancer cells under normoxic (21% O₂) conditions

Complex compound of formula Id

| λ (nm) | IC₅₀ (nM) | PI |
|---|---|---|
| Dark | >10⁵ | — |
| 540 nm | 11 ± 5 | >9090 |

TABLE 4
Phototoxicity of complex compound of formula
If towards CT-26 cancer cells under normoxic (21%
O₂) and hypoxia (2% O₂) conditions

| Complex compound of formula If | |
|---|---|
| Normoxia (21% O₂) | Hypoxia (2% O₂) |

| λ (nm) | IC₅₀ (nM) | PI | IC₅₀ (nM) | PI |
|---|---|---|---|---|
| Dark | >10⁵ | — | >10⁵ | — |
| 540 nm | 13 ± 3 | >7692 | 75 ± 5 | >1333 |
| 620 nm | 92 ± 30 | >1086 | 160 ± 39 | >625 |

TABLE 5
Phototoxicity of complex compound of formula Im towards
CT-26 cancer cells under normoxic (21% O₂) conditions.

Complex compound of formula Im

| λ (nm) | IC₅₀ (nM) | PI |
|---|---|---|
| Dark | >10⁵ | — |
| 770 nm | 820 ± 20 | >122 |

TABLE 6
Phototoxicity of complex compound of formula Ic towards human cancer cells.

| λ | CT-26 | | HT-29 | | A-549 | | A-2780 | |
|---|---|---|---|---|---|---|---|---|
| (nm) | IC₅₀ (nM) | PI | IC₅₀ (nM) | PI | IC₅₀ (nM) | PI | IC₅₀ (nM) | PI |
| dark | >2.5 × 10⁵ | — | >2.5 × 10⁵ | — | >2.5 × 10⁵ | — | >2.5 × 10⁵ | — |
| 740 | 760 ± 60 | >329 | 350 ± 40 | >714 | 990 ± 4 | >252 | 2970 ± 130 | >84 |

TABLE 7
Phototoxicity of complex compound of formula
Ic towards human normal cells.

| λ | RPE-1 | | HEK-293 | | MRC-5 | |
|---|---|---|---|---|---|---|
| (nm) | IC₅₀ (nM) | PI | IC₅₀ (nM) | PI | IC₅₀ (nM) | PI |
| dark | >10⁵ | — | >10⁵ | — | >10⁵ | — |
| 740 | 800 ± 180 | >125 | 3330 ± 900 | >30 | 490 ± 30 | >205 |

TABLE 8
Phototoxicity of complex compound of the indicated formulas towards
CT-26 cancer cells under normoxic (21% O₂) and hypoxic (2% O₂).

| | Normoxia (21% O₂) | | | | | Hypoxia (2% O₂) | | |
|---|---|---|---|---|---|---|---|---|
| | Dark | 740 nm | | 770 nm | | Dark | 740 nm | |
| | IC₅₀ | IC₅₀ | | IC₅₀ | | IC₅₀ | IC₅₀ | |
| | (μM) | (μM) | PI | (μM) | PI | (μM) | (μM) | PI |
| Im | >100 | 0.17 ± 0.02 | >588 | 0.82 ± 0.02 | >122 | >100 | 0.60 ± 0.22 | >168 |
| In | >100 | 0.86 ± 0.03 | >116 | 8.70 ± 1.08 | >11 | >100 | 3.72 ± 0.96 | >27 |
| Io | >100 | 7.95 ± 1.7 | >13 | 13.19 ± 1.97 | >8 | >100 | 25.14 ± 1.45 | >4 |
| IVa | >100 | 1.51 ± 0.44 | >66 | 2.58 ± 0.50 | >39 | >100 | 3.22 ± 0.87 | >31 |

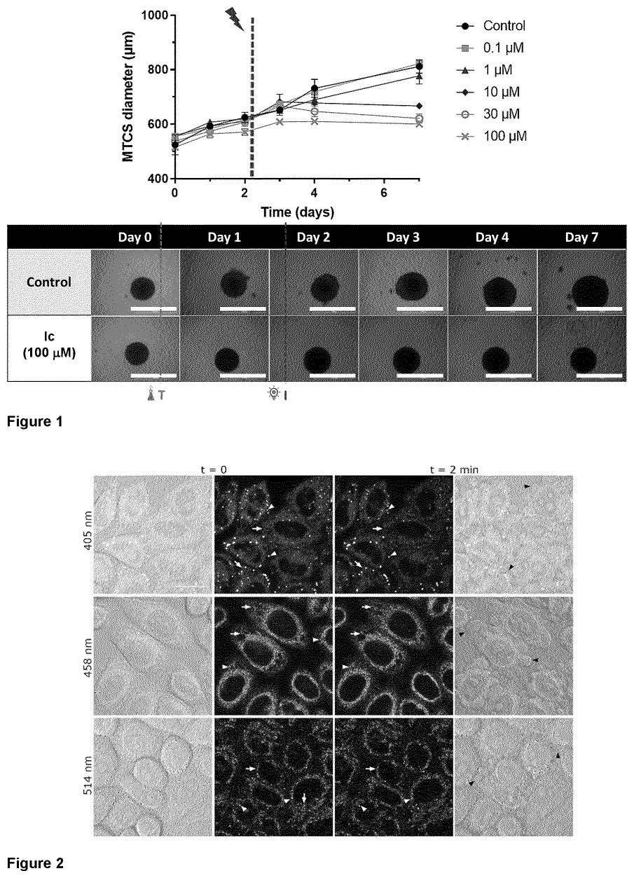

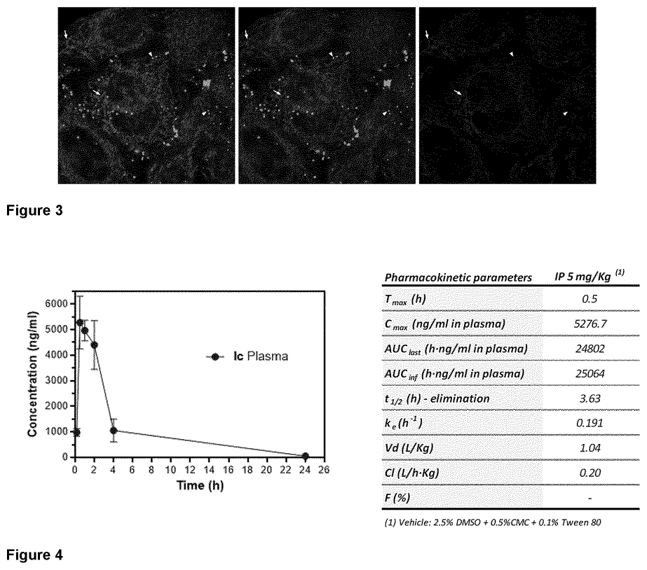

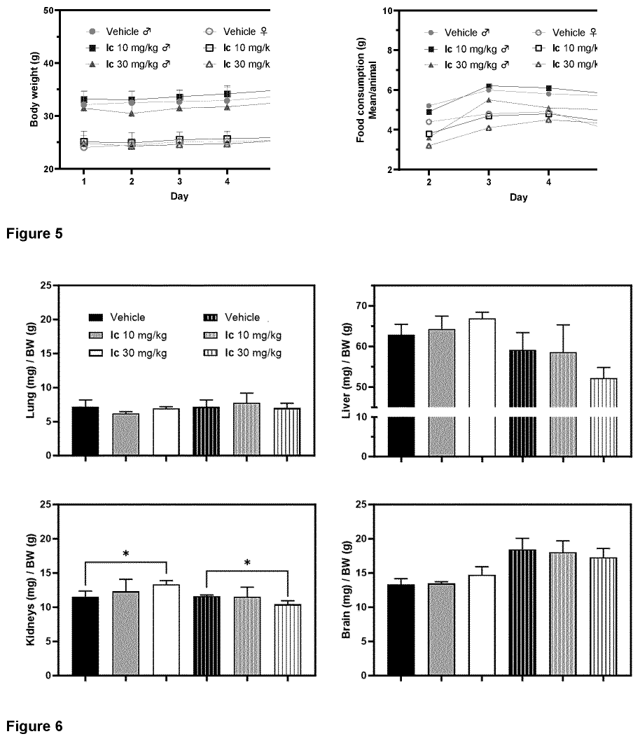

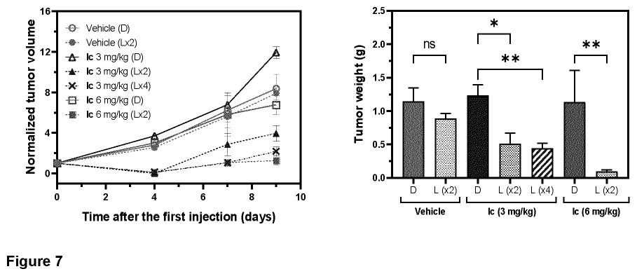

In addition, it is noted that complex compound Ic was able to inhibit the growth of 3D tumor spheroids (FIG. 1), having mitochondria as a specific subcellular target (FIGS. 2 and 3). In vivo studies of Ic have demonstrated a good biodistribution profile and excellent safety. As shown in FIG. 4, the maximum concentration of compound in mouse plasma was reached at 30 min post-administration (5 mg/kg), and the compound was completely eliminated after 24 h. In toxicological maximum tolerated dose (MTD) study, all parameters of the treated animals (male and female albino swiss CD1 mice) at the two investigated concentrations (10 and 30 mg/kg) were comparable to those of the control group (FIGS. 5 and 6): body weight, body weight increase, food consumption, organ weight, gross necropsy, haematology and biochemistry analysis. No mortality and no clinical signs were recorded in the vehicle and Ic-treated male and female experimental groups during the observation period (5 days). In addition, complex compound Ic was able to reduce tumor growth in a murine subcutaneous colorectal cancer model (FIG. 7).

1. A compound of formula I, including any stereoisomer or E/Z isomer thereof, 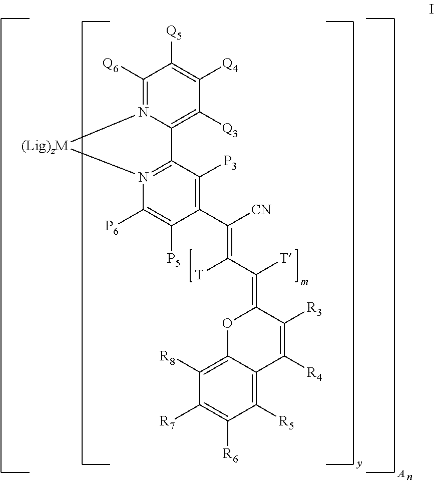
wherein:
M is a metal cation selected from the group consisting of: Fe²⁺, Ru²⁺, Os²⁺, Co³⁺, Rh²⁺, Rh³⁺, Ir³⁺, Ni²⁺, Pd²⁺, Pt²⁺, and Pt⁴⁺;
y=1, 2, or 3; and z=0, 1, or 2; on the proviso that y+z=2 when M is Ni²⁺, Pd²⁺ or Pt²⁺, and y+z=3 when M is Fe²⁺, Ru²⁺, Os²⁺, Co³⁺, Rh²⁺, Rh³⁺, Ir³⁺, or Pt⁴⁺;
m=0, 1, 2, 3, or 4;
radicals T and T′ at each occurrence are each one a radical independently selected from the group consisting of: H and (C1-C3)-alkyl;
radicals P₃, P₅, P₆, Q₃, Q₄, Q₅, Q₆, R₃, R₅, R₆, and R₈ at each occurrence are each one a radical independently selected from the group consisting of: H, (C1-C3)-alkyl, (C3-C6)-cycloalkyl, CH═CHR, CF₃, CHF₂, CH₂F, CF₂CF₃, F, Cl, Br, I, OR, C(═O)OR, O(C═O)R, C(═O)NR, NRC(═O)R′, NRR′, phenyl, mono-substituted phenyl, di-substituted phenyl, and tri-substituted phenyl; wherein substituents on the phenyl ring are attached to any of the possible substitution positions, and they are independently selected from the group consisting of: F, Cl, Br, I, NO₂, (C1-C3)-alkyl, OH, O[(C1-C3)-alkyl], NH₂, NH[(C1-C3)-alkyl], and N[(C1-C3)-alkyl]2; wherein when m>1 and radical R₃ is CH₂ a 6-membered ring may be formed between the CH₂ of R₃ and the T′ radical closest D the coumarin backbone provided that T′=CH₂, thus providing a compound of formula IV;
radical R₇ is H, NO₂, OR, NRR′, N(CH₂COOH)₂, N(CH₂CH₂SO₃H)₂, N(CH₂CONHCH₂CH₂NMe₂)₂, or: an unsubstituted, a mono-(R)-substituted, a di-(R,R′)-substituted, or a tri-(R,R′,R″)-substituted radical of each one of the heterocyclic radicals 1-aziridinyl, 1-azetidinyl, 1-pyrrolidinyl, I-piperidinyl, 4-morpholinyl, or 1-piperazinyl; alternatively R₆, R₇, and R₈ form together the bicycle system of the accompanying formula, wherein V₁, V₂, V₃, V₄, V₅, V₆, V₇, V₈, V₉, V₁₀, V₁₁, and V₁₂ are each one a radical independently selected from the group consisting of H and (C1-C3)-alkyl; 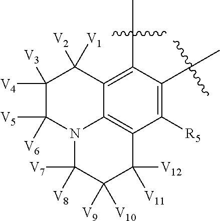
radical R₄ is H, (C1-C3)-alkyl, (C3-C6)-cycloalkyl, CF₃, CHF₂, CH₂F, CF₂CF₃, F, Cl, Br, I, OR, C(═O)OR, O(C═O)R, C(═O)NR, NRC(═O)R′, NRR′, phenyl, a mono-(R)-substituted phenyl, a di-(R, R′)-substituted phenyl, a tri-(R, R′, R″)-substituted phenyl, or a radical CE₁E₂E₃; wherein: E₁ and E₂ are radicals independently selected from the group consisting of: H, F, Cl, Br, I, OR, NRR′, NO₂, (C1-C3)-alkyl, phenyl, and a mono-(R)-substituted phenyl; and E₃ is OH, O[(C1-C3)-alkyl], or a radical of the accompanying formula wherein p=0, 1, 2, 3 or 4; and D₁ and D₂ at each occurrence are radicals independently selected from the group consisting of H, O[CH₂]_{q}—X, S[CH₂]_{q}—X, and N[[CH₂]_{q}—X]₂, being q=1 or 2, and X=F, Cl, Br or I. 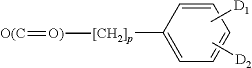
or a radical of the accompanying formula wherein p=1, 2, 3 or 4; and D3 is a radical independently selected from the group consisting of H, H(C═O), [(C1-C3)-alkyl](C═O), (OH)₂P(═O)O—CH₂—O(C═O), or (PhO)(OH)P(═O) 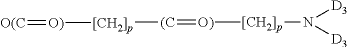
Lig at each occurrence is a bidentate ligand independently selected from the group consisting of ligands of accompanying formulae: Lig 1, Lig 2, Lig 3, Lig 4, Lig 5, Lig 6, Lig 7, Lig 8, Lig 9, Lig 10, Lig 11, Lig 12, and Lig 13; wherein G is either an N atom, or a C atom with one negative charge that is formed in situ from a C—H when a H⁺ is lost in the formation of the carbon-metal bond; and wherein R₁₀, R₁₁, R₁₂, R₁₃, R₁₄, R₁₅, R₁₆, R₁₇, R₁₈, R₁₉, R₂₀ and R₂₁ at each occurrence is each one a radical independently selected from the group consisting of: H, (C1-C3)-alkyl, phenyl, and mono-, di- and tri-substituted phenyl, substituents attached to the phenyl ring being independently selected from F, Cl, Br, I, OR and NRR′; and 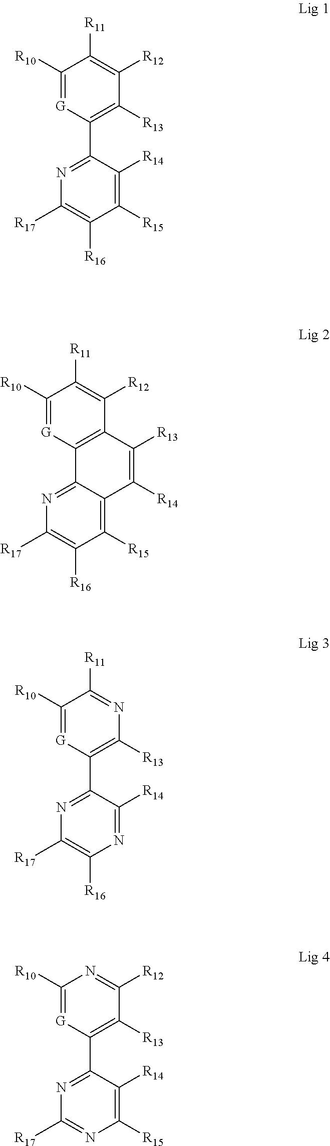 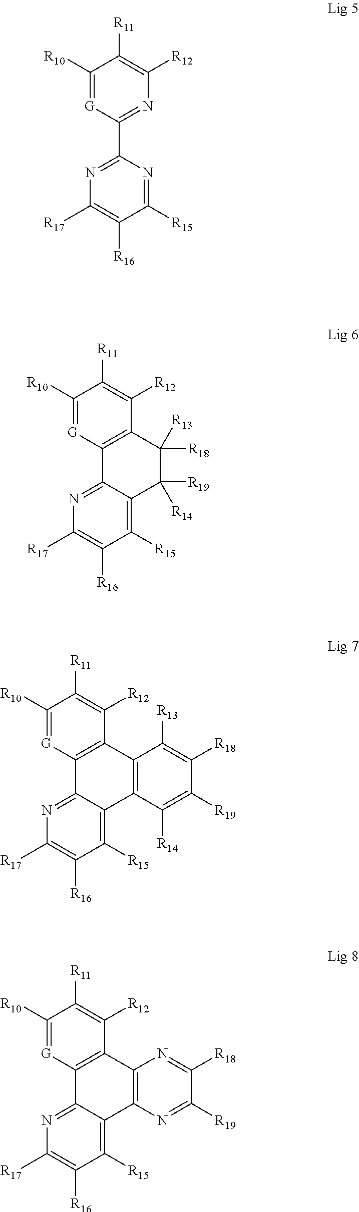 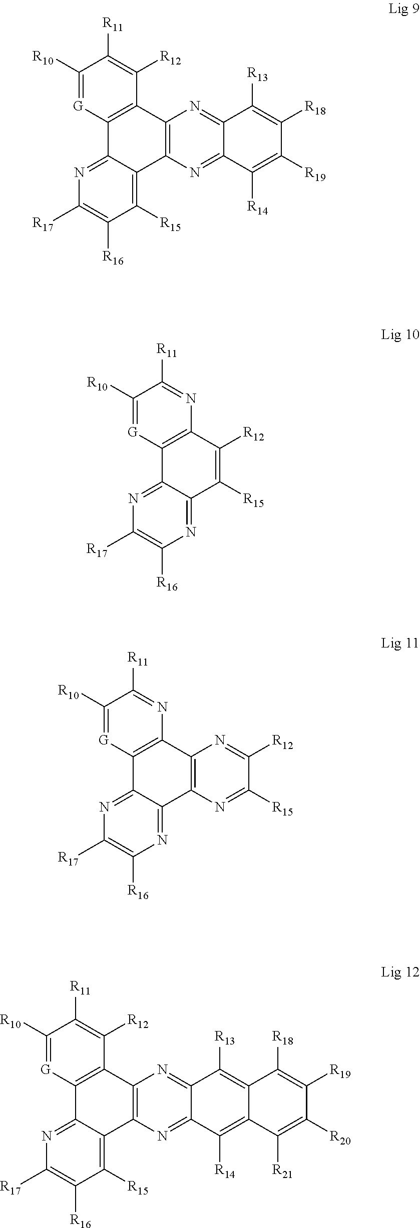 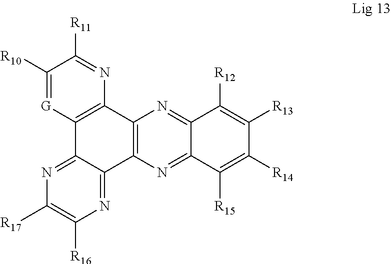
A is an anion from a pharmaceutically acceptable acid; n being an integer or fractional number whereby the compound of formula I is electrically neutral;
wherein R, R′ and R″ at each one of the above-mentioned occurrences are each one a radical independently selected from the group consisting of: H, F, Cl, Br, I, NO₂, (C1-C3)-alkyl, OH, O[(C1-C3)-alkyl], NH₂, NH[(C1-C3)-alkyl], and N[(C1-C3)-alkyl]₂.

2. The compound according to claim 1, wherein the anion A is selected from the group consisting of: Cl⁻, Br⁻, PF₆⁻, PF₄⁻, BF₄⁻, ClO₄⁻, CF₃SO₃⁻, SO₄²⁻, CF₃COO⁻, acetate, formate, and oxalate.

3. The compound according to claim 2, wherein the anion A is Cl⁻.

4. The compound according to claim 1, wherein y=1, z=2, and the metal cation M is Fe²⁺, Ru²⁺, Os²⁺, Co³⁺, Rh²⁺, Rh³⁺, Ir³⁺, or Pt⁴⁺.

5. The compound according to claim 1, wherein the bidentate ligand Lig is Lig 1 or Lig 2; and each one of the radicals R₁₀, R₁₁, R₁₂, R₁₃, R₁₄, R₁₅, R₁₆, R₁₇, R₁₈, R₁₉, R₂₀ and R₂₁ at each occurrence is independently selected from the group consisting of H, methyl and phenyl.

6. The compound according to claim 1, wherein G is N.

7. The compound according to claim 6, wherein the bidentate ligand Lig is 2,2′-bipyridine (bpy) or 4,7-diphenyl-1,10-phenanthroline (BPhen).

8. The compound according to claim 1, wherein each one of the radicals P₃, P₅, P₆, Q₃, Q₄, Q₅, Q₆, R₃, R₅, R₆, and R₈ at each occurrence is independently selected from the group consisting of H and (C1-C3)-alkyl; and the radical R₇ is H, N[(C1-C3)-alkyl)]₂ or NO₂; alternatively the radicals R₆, R₇ and R₈ form together the bicycle system of the formula shown in claim 1, wherein each one of V₁, V₂, V₃, V₄, V₅, V₆, V₇, V₈, V₉, V₁₀, V₁₁, and V₁₂ is H.

9. The compound according to claim 1, wherein the radical R₄ is H, (C1-C3)-alkyl, CF₃, CH(CH₃)OH, CH(CH₃)O(C═O)—[CH₂]₃-Ph, or the radical of accompanying formula. 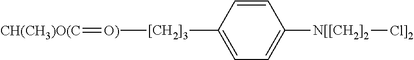
or radical of formula: CH(CH₃)O(C═O)—[CH₂]₂—(C═O)—CH₂—NH₂.

10. The compound according to claim 1, wherein m=0, 1 or 2.

11. The compound according to claim 1, wherein the metal cation M is Ru²⁺.

12. The compound according to claim 11, wherein formula I is selected from the group consisting of accompanying formulas Ia, Ib, Ic, Id, le, If, Ig, Ih, Im, In, Io, Ip, Iq, IVa and IVb:

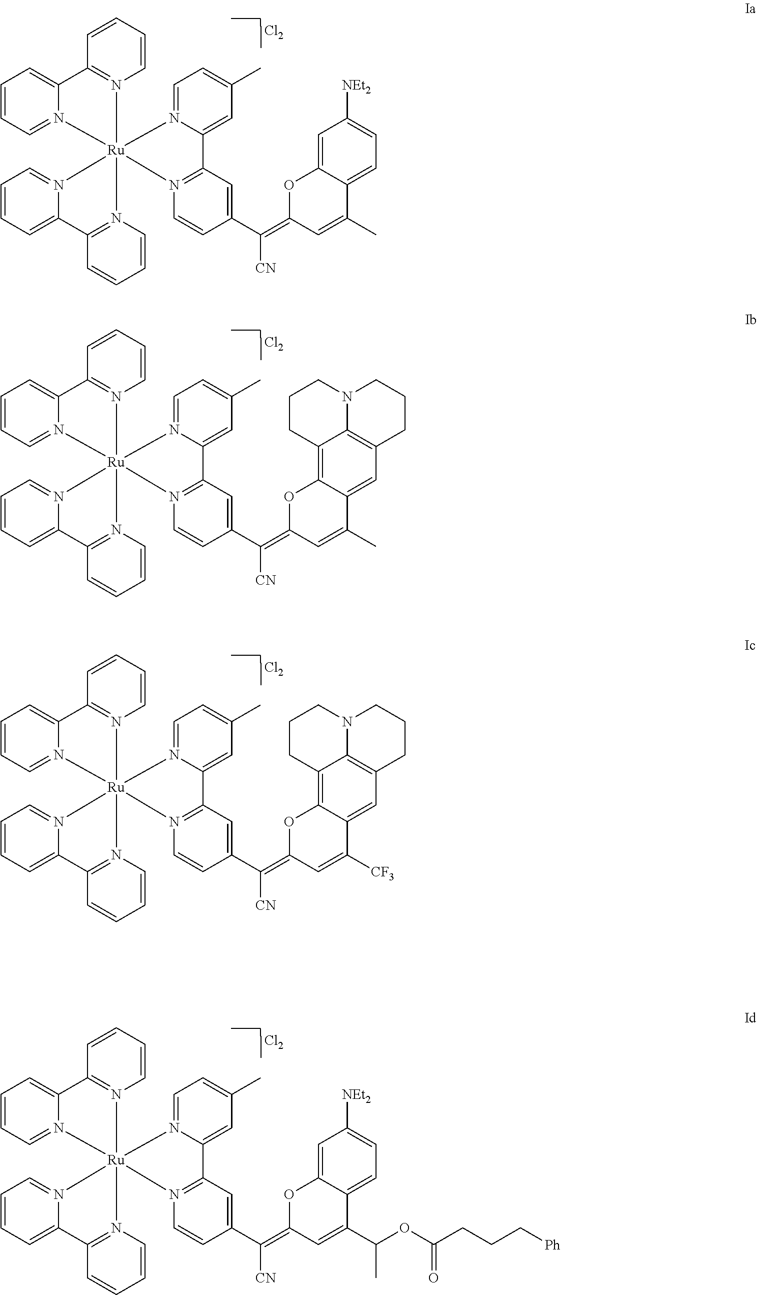

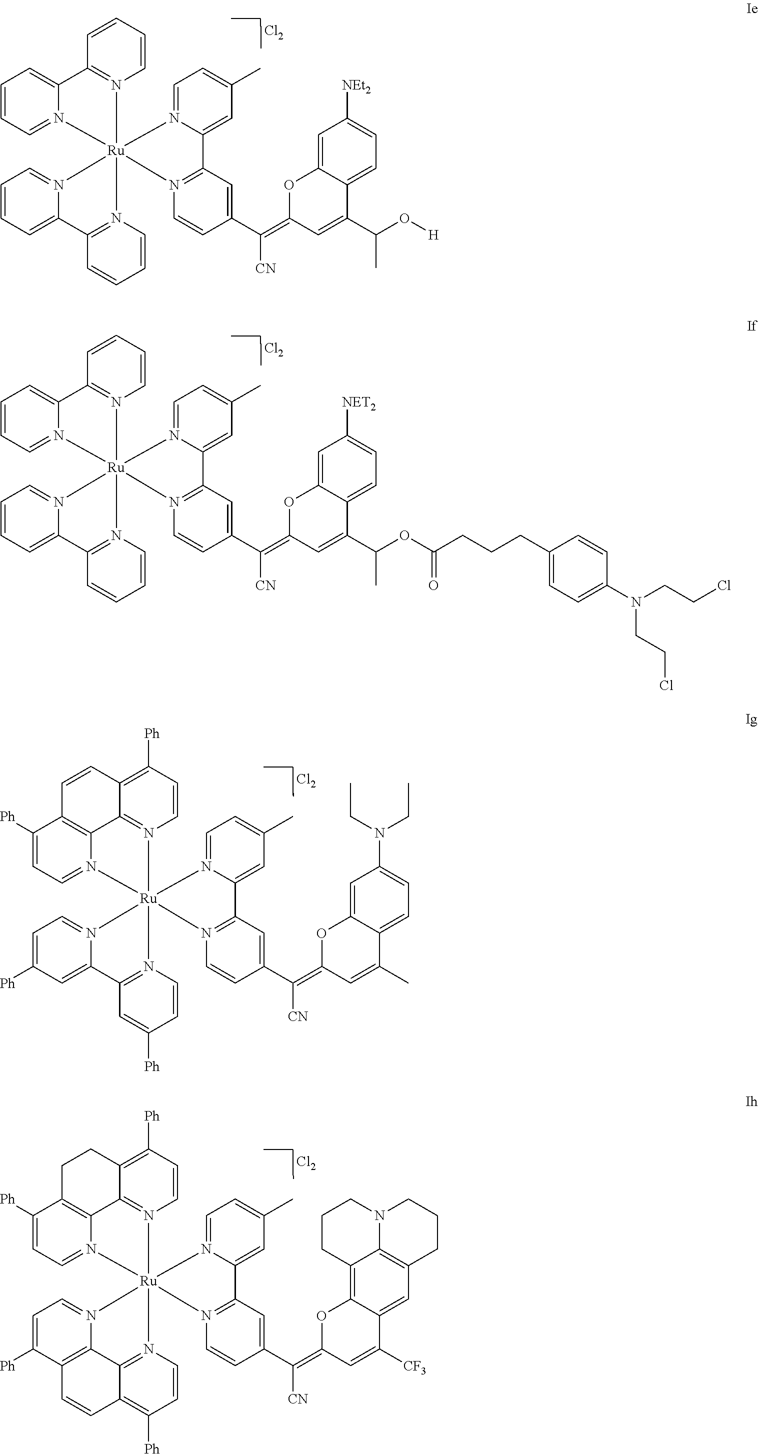

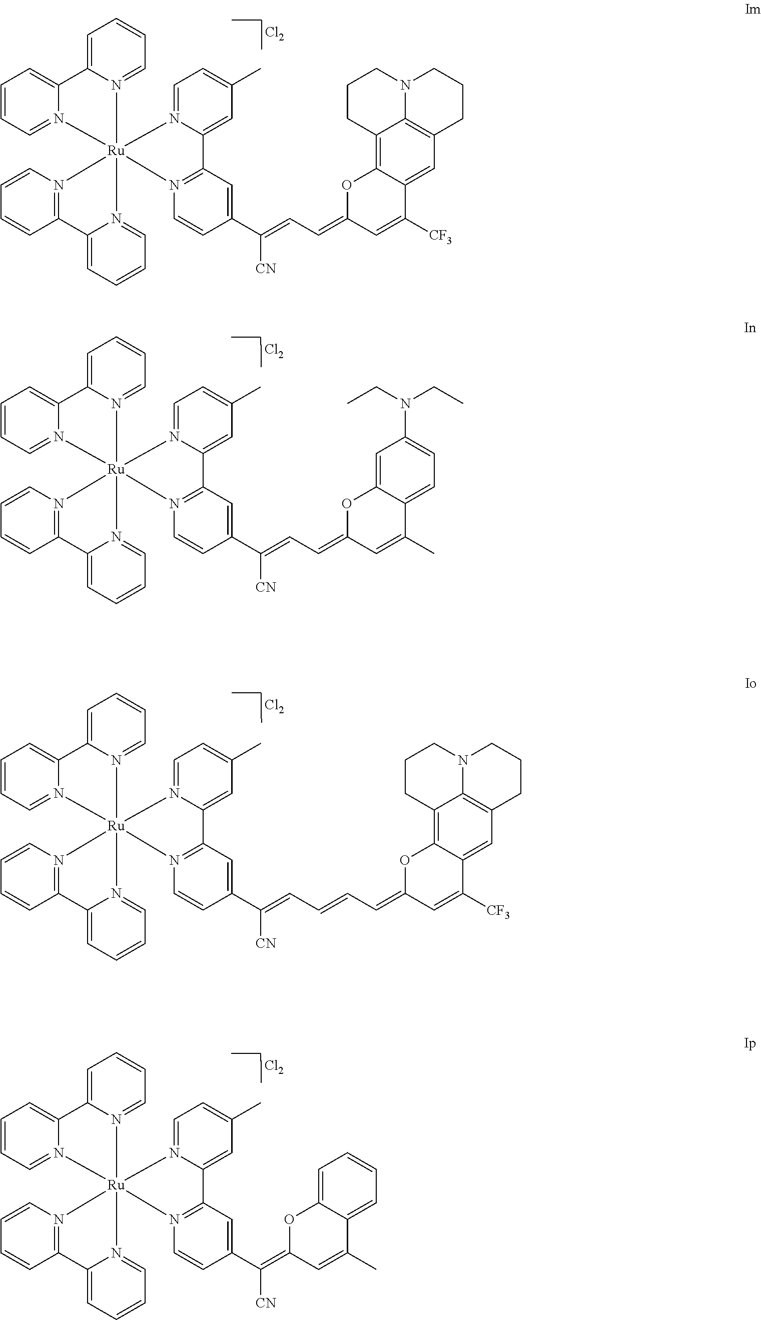

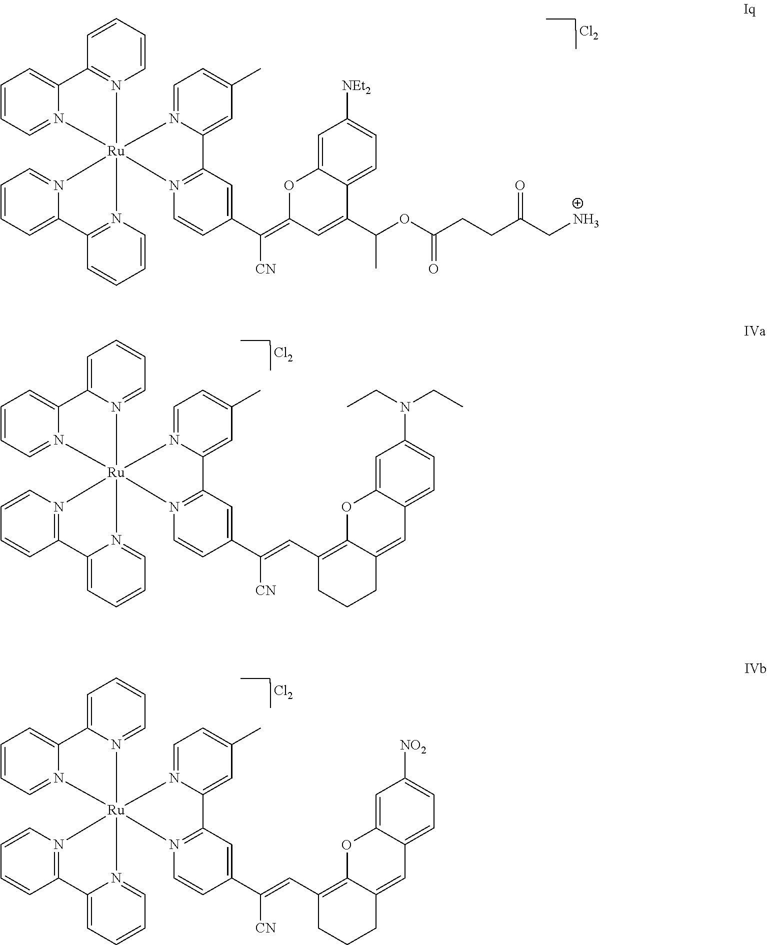

13. A compound of formula II, including any stereoisomer or E/Z isomer thereof, 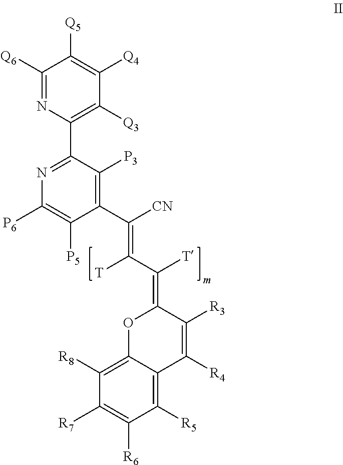
wherein m, P₃, P₅, P₆, Q₃, Q₄, Q₅, Q₆, R₃, R₄, R₅, R₆, R₇, R₈, T and T′ are as defined in claim 1.

14. The compound according to claim 13, wherein formula II is selected from the group consisting of accompanying formulas IIa, IIb, IIc, IId, IIe, IIf, IIm, IIo, IIn, IIp, IIq, IIr, Va, and Vb:

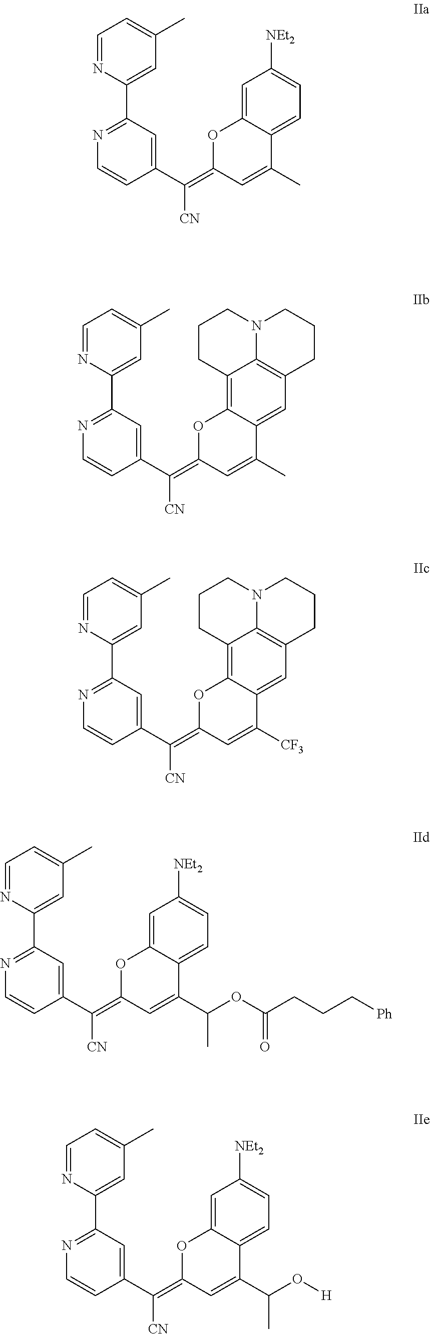

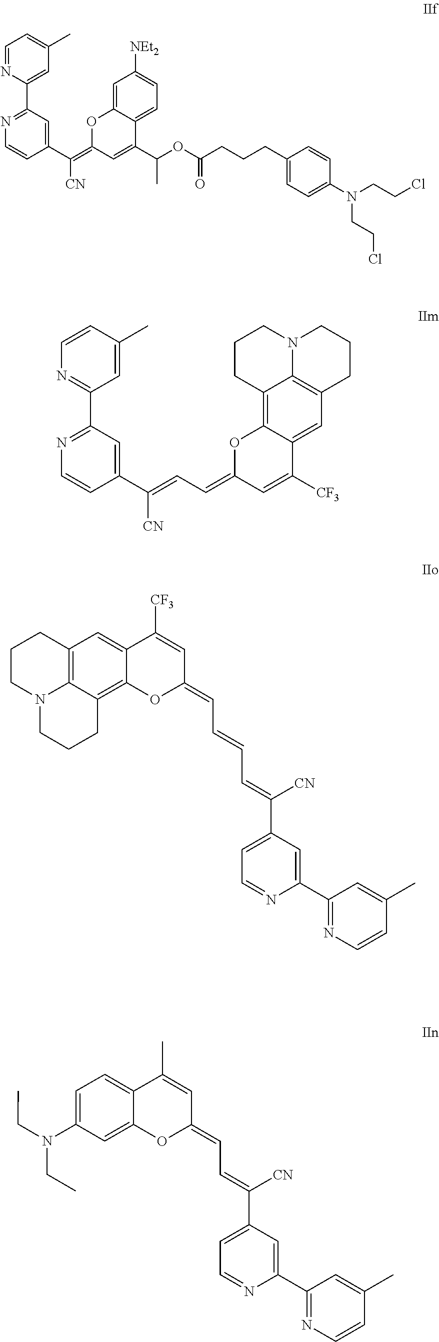

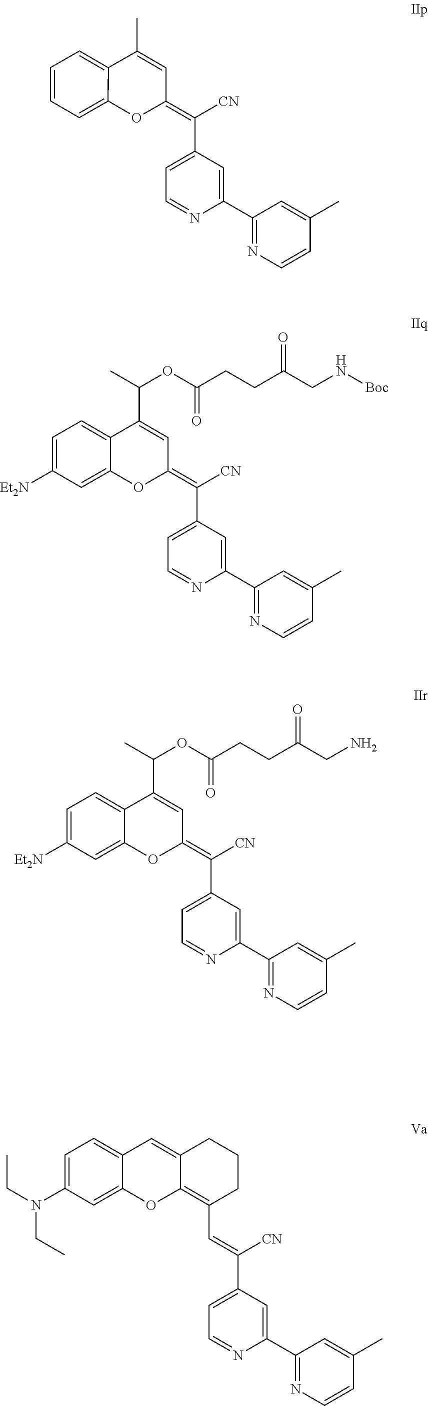

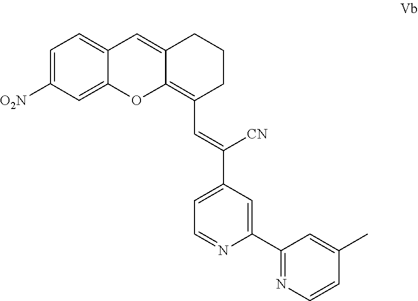 [SMILES: Cc1ccnc(-c2cc(/C(C#N)=C/C3=C4Oc5cc([N+](=O)[O-])ccc5C=C4CCC3)ccn2)c1]

15. A pharmaceutical composition comprising a therapeutically effective amount of a compound of formula I as defined in claim 1, together with appropriate amounts of excipients, carriers or vehicles.

16. A method of treating a human subject, comprising administering the compound of formula I as defined in claim 1.

17. The method of claim 16, wherein the method comprises administering the compound as photosensitizer in photodynamic therapy of a human condition.

18. The method according to claim 17, wherein the human condition is a cancer, a skin condition, a fungal infection, or a microbial infection.
